(19) United States
(12) Patent Application Publication
(10) Pub. No.: US 2012/0123326 A1
(43) Pub. Date: May 17, 2012

(54) CATHETER SYSTEMS WITH DISTAL END FUNCTION, SUCH AS DISTAL DEFLECTION, USING REMOTE ACTUATION OR LOW INPUT FORCE

(76) Inventors: Steven C. Christian, New Brighton, MN (US); Jeffrey A. Schweitzer, St. Paul, MN (US)

(21) Appl. No.: 13/091,552

(22) Filed: Apr. 21, 2011

Related U.S. Application Data
(60) Provisional application No. 61/413,169, filed on Nov. 12, 2010.

(51) Int. Cl. A61M 25/092; A61B 18/18

(52) U.S. Cl. 604/95.03; 604/95.01; 604/95.05; 606/33

(57) ABSTRACT

Systems capable of providing force and displacement outputs sufficient to actuate remote mechanisms to enhance catheter capabilities include both low-force and remote actuation arrangements. The remote actuation and low-force actuation systems may be used for catheter distal end deflection, sensor deployment, feedback controlled movement, fluid delivery rate and directional control applications as well as catheter retention mechanism deployment. Remote actuation mechanism may employ phase change based, magnetic based or hydraulic based. Low-force remote actuation structures include a coaxially-extending pull wire, a reaction member, and a remote mechanism responsive to the pull force.

### CROSS-REFERENCE TO RELATED APPLICATIONS

This application claims the benefit of U.S. provisional application No. 61/413,169, filed 12 Nov. 2010, which is hereby incorporated by reference in its entirety as though fully set forth herein.

### BACKGROUND OF THE INVENTION

a. Field of the Invention

The present invention relates generally to catheter systems for diagnostic and therapeutic purposes, and more particularly to catheter systems with distal end functionality, such as distal deflection, by using remote actuation or low input force.

b. Background Art

Many medical procedures require the introduction of specialized medical devices in the body, for example, in and around the human heart. Such specialized devices include introducers, access sheaths, catheters, dilators, needles, and the like. Such devices may be used to access areas of the body, for example areas of the heart such as the atria or ventricles, and have been used in such medical procedures for a number of years. During such a procedure, a physician typically maneuvers the device through the vasculature of a patient to the desired location, such as the heart where, for example, the physician may explore the chambers and locate sites for a diagnostic or a therapeutic function (e.g., ablation). Accordingly, such devices preferably exhibit at least some degree of flexibility (e.g., deflection or bending capability) to allow for such maneuvering.

To achieve the foregoing, pull wires may be provided, which are used to control the movement and relative curvature of the device. Pull wires extend generally along the length of the device (i.e., typically within an outer wall), and may be coupled at the distal end to a pull ring and at the proximal end to a control mechanism. A typical control mechanism may be, for example, a user-actuated knob that can be rotated, which in turn “pulls” one or more of the pull wires in a predetermined fashion, resulting in the desired deflection. Forces transmitted through the pull wires act to deflect the softer distal portions of the device. The tensile forces, however, can be significant, and must be supported by the main body of the catheter on its path to the deflectable distal section.

As shown in FIG. 1, the amount of force required to deflect a catheter's distal tip increases as the degree of deflection increases. The force must be carried by the main body of the catheter. Since the catheter must, at the same time, be able to negotiate a tortuous path through the patient, the flexibility of the catheter body must be balanced against (i.e., limited by) the need to support the tensile pull wire forces. In other words, while increasing the stiffness of the catheter body (e.g., shaft) may be desirable because of the increased ability to handle pull wire forces, this same increase in stiffness will reduce the catheter's flexibility, diminishing the ability of the catheter to bend/deflect as needed for navigation through the patient's vasculature. Additionally, situations that would benefit the most from softer, more flexible materials (e.g., increased deflection angles) are also those involving the highest pull forces. These conflicting requirements thus create challenges in catheter design, which affect tactile feedback and catheter manipulation within the heart.

In addition, during use, the physician must be able to smoothly rotate the catheter (i.e., about its main axis), while deflected and constrained within vascular sheaths, in order to locate ablation sites. The pull wire force mentioned above, in some circumstances, limits ability of the catheter to rotate. For example, while un-deflected (i.e., in the absence of pull wire tensile forces), a steerable catheter contained within an introducer may have sufficient clearance to allow rotation of the catheter within the sheath. However, when the pull wire is placed in tension, the force carried by the body of the catheter may cause the catheter to bend enough to become “locked” within the introducer (i.e., any pre-existing rotational clearance is eliminated), thereby preventing rotation of the catheter.

Additionally, repeated deflection of the catheter shaft using pull wires may cause a compression (i.e., an axial foreshortening) of the catheter shaft at the distal end where the deflection occurs. The compression causes the catheter distal end to lose its original shape, dimensions and deflectability.

There is therefore a need for a catheter system that eliminates or minimizes one or more of the problems identified above.

### BRIEF SUMMARY OF THE INVENTION

One advantage of the methods and apparatus described, depicted and claimed herein relates to the reduction and/or elimination of pull wire system effects, for example, reducing or eliminating pull wire forces resolved through the catheter body. Another advantage of the methods and apparatus described, depicted and claimed herein is that such remote actuator systems will provide greater precision (i.e., greater control and feedback). A still further advantage of the methods and apparatus described, depicted and claimed herein involve a relaxation of the construction requirements of a catheter body and/or a shaft design (i.e., conventional designs must strike a balance to accommodate both expected tension in the catheter body as well as the need for increased flexibility to allow for navigation through tortuous paths).

A catheter, in an embodiment, includes a shaft, an actuator, and a controller. The shaft includes a distal end portion and a remainder portion that in turn includes a proximal end portion. The actuator is disposed at the distal end portion of the shaft and is configured to produce a controlled movement in response to an actuation input. The controller is configured to produce such an actuation input. The controller, in an embodiment, is configured to be disposed in a location that is remote from the distal end portion of the shaft (i.e., the location where the actuator is disposed). The actuation input is communicated to the distal, remote actuator without altering the mechanical characteristics of the remainder portion of the shaft.

In an embodiment, the actuator comprises a material phase change actuator, although in other embodiments the actuator may be any one of a magnetic actuator, a material phase change actuator, a hydraulic actuator, a piezo-electric actuator, an electric actuator, and a combination of any of the foregoing types of actuators. In an embodiment where the actuator is a material phase change-based actuator, such actuator may comprise at least one shape memory alloy (SMA) coil or wire form, for example, a coil or wire form comprising Nitinol material (NiTi) configured to transition, in response to the actuation input, from a first state to a second, different state having a different physical or mechanical configuration. In an embodiment, the first state may be a compressed condition (shortened length) while the second state may be an extended condition (elongated length).

In further embodiments, the controlled movement caused by the actuator is configured to facilitate at least one of a number of uses: (i) a deflection of the distal end portion of the shaft, (ii) for sensor deployment, (iii) for control of a fluid delivery rate, (iv) for control of a fluid delivery direction, and (v) for deployment of a catheter retention mechanism. For fluid delivery rate and direction control, the actuator may be configured to control a fluid valve.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a chart showing pull wire force as a function of distal end deflection (in degrees).

FIG. 2 is a diagrammatic side view of a catheter, in a first embodiment, having remote actuation.

FIGS. 3A-3B are end and side (cross-sectional) views, respectively, of a first deflection mechanism embodiment.

FIGS. 4A-4B are end and side (cross-sectional) views, respectively, of a second deflection mechanism embodiment.

FIGS. 5A-5B are side views of a V-shaped spring, suitable for use in the deflection mechanisms of FIGS. 3A-3B and FIGS. 4A-4B, in uncompressed and compressed conditions, respectively.

FIGS. 6A-6B are end and side (cross-sectional) views, respectively, of a third deflection mechanism that further includes a passive, resilient member to provide a restorative force.

FIG. 7 is a side view of a fourth deflection mechanism embodiment coupled to a hydraulic remote actuator.

FIGS. 8A-8B are side views of a fifth deflection mechanism embodiment in the form of a bellows assembly, in both retracted and extended conditions, respectively.

FIG. 9 is a diagrammatic view of a catheter having remote actuation in the form of a plurality of remote actuators, each having a respective working length.

FIGS. 10A-10B are side and top diagrammatic views, respectively, showing an exemplary use of an ablation catheter having a remote actuator adapted for creating a linear lesion.

FIGS. 11-12 are cross-sectional and diagrammatic views, respectively, illustrating a coaxially-disposed pull wire embodiment for a catheter that features remote actuation that does not alter the mechanical characteristics of the catheter body.

FIG. 13 is a simplified, cross-sectional view of a further coaxially-extending pull wire embodiment, for a catheter.

FIG. 14 is a side view of a still further coaxially-extending pull wire embodiment, for a catheter.

FIGS. 15-16 show a still further coaxially-extending pull wire embodiment, for a catheter, having a diagonally oriented spar member, in various stages of manufacture.

FIGS. 17-18 illustrate a catheter embodiment similar to the embodiment illustrated in FIGS. 15-16, but with a tapered spring substituted for a straight spring.

FIG. 19 is a pull wire embodiment for a catheter similar to the embodiment illustrated in FIGS. 15-16, except that the spar member is axially oriented and offset from a main axis.

FIG. 20 is an embodiment similar to the embodiment of FIG. 19, but including an on-axis spar in combination with a pair of pull wires, adapted for bi-directional deflection.

FIG. 21 is an embodiment, similar to the embodiment of FIG. 19, but in which the coaxially-extending pull wire has been replaced by a remotely-disposed electric solenoid actuator.

FIG. 22 is a coaxially-extending pull wire embodiment for a catheter in which the deflection mechanism is axially movable.

FIGS. 23-26 are isometric views of a catheter embodiment having a remotely-actuated irrigation fluid valve, in closed (FIGS. 23-24) and open (FIGS. 25-26) positions, respectively.

FIG. 27 is an isometric view of a remote actuator having counter-acting shape memory alloy (SMA) coils.

FIG. 28 is an isometric view of a remote actuator having a single acting SMA coil with a pull wire reset.

FIGS. 29A-29C are isometric views showing a remote actuator having a single acting SMA coil, in progressively deflected conditions.

FIGS. 30-31 are isometric views of a catheter embodiment having a remote actuator configured for three-dimensional electrode array deployment, in retracted and deployed positions, respectively.

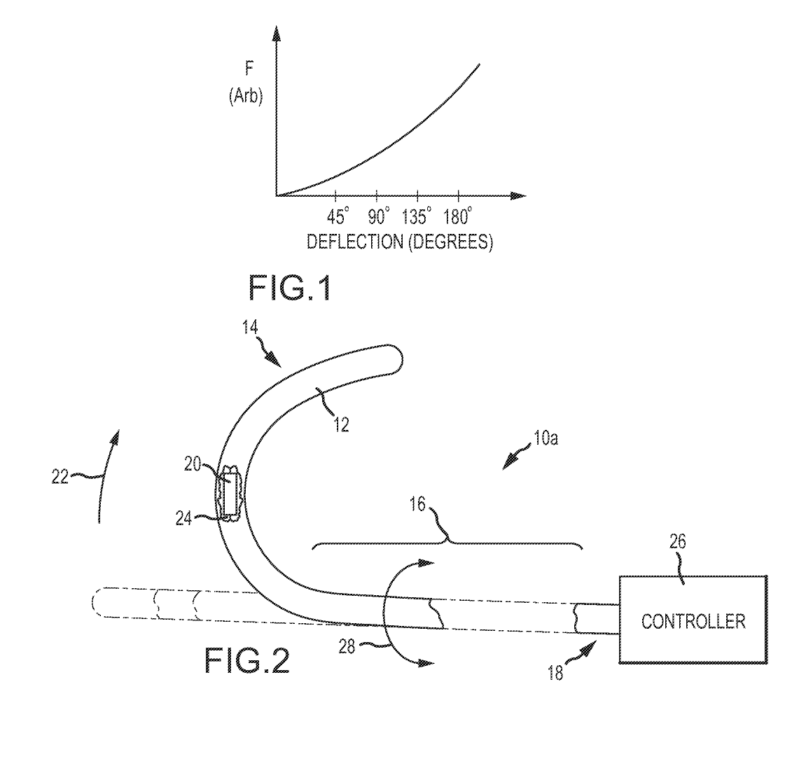

### DETAILED DESCRIPTION OF THE INVENTION

The instant disclosure describes alternatives to conventional pull wire mechanisms capable of providing force and displacement outputs sufficient to actuate remote mechanisms and potentially enhance catheter capabilities. The embodiments described herein address the shortcomings of conventional pull wire arrangements by providing a means or mechanism to activate various catheter distal tip functions. For example, such means or mechanisms involve either distally-located actuators or a centrally-disposed (i.e., coaxial with the catheter body), relatively low force pull wire arrangement. Embodiments described herein may be used for a variety of useful purposes including, without limitation, (i) deflecting a catheter in a controlled manner without using pull wires, or in the case of a coaxial pull wire, using only a relatively low force pull wire, (ii) sensor deployment, (iii) feedback controlled movement, (iv) fluid delivery rate and directional control, (v) deployment of catheter retention mechanisms, and the like. Embodiments described herein, particularly remotely-located actuator-based embodiments, enable the use of such means or mechanisms across a plurality of different catheter configurations, since their function is not highly dependent on the particular catheter configuration.

In addition, embodiments described herein provide greater precision and catheter control, and otherwise enable additional functions. In the case of remote distal end deflection, the embodiments described herein eliminate or reduce adverse pull wire system effects (e.g., shaft compression, need for design trade-offs between shaft flexibility for maneuvering and shaft stiffness to accommodate pull forces, etc.) that would otherwise alter the mechanical characteristics of the catheter body (e.g., shaft). In addition to improved catheter control, embodiments according to the invention provide for reduced electrophysiological (EP) procedure times, improved ablation capabilities, improved tactile feedback, predictable deflection performance and greater coverage of the endocardial surface.

As alluded to above, embodiments of the invention include further benefits with respect to manufacturing, such benefits including a reduced cost of manufacture (i.e., due to simplified designs compared to conventional designs), less dependence on particular catheter material characteristics in the catheter design, additional deflection characteristics, and a simplified handle design (e.g., no need for a complicated pull wire(s) mechanisms), to describe a few. Remote or low-force deflection arrangements according to the invention may also benefit the development of more advanced EP procedures.

Referring now to FIG. 2, a catheter 10a includes a shaft 12 having a distal end portion 14 and a remainder portion 16 that in turn includes a proximal end portion 18. Catheter 10a further includes an actuator 20 disposed at distal end portion 14 of shaft 12. Actuator 20 is configured to produce a controlled movement. It should be understood that catheter 10a, other than the inventive structures and functionality described herein, may otherwise generally incorporate conventional materials and construction approaches. For example only, shaft 12 may be fabricated according to known processes, such as multilayer processing including extrusion processes, mandrel-based processes and combinations thereof from any suitable biocompatible polymer material known in the art of medical instruments, such as engineered nylon resins and plastics, including but not limited an elastomer commercially available under the trade designation PEBAX® from Arkema, Inc. of a suitable durometer, melting temperature and/or other characteristics Likewise, various other components, unless otherwise stated, may generally be formed using any suitable biocompatible polymer material known in the art of medical instruments, such as engineered nylon resins and plastics or likewise any suitable biocompatible metal material, such as stainless steel, platinum, nickel titanium alloys and the like.

As shown in FIG. 2, remote actuator 20 is responsive to an actuation input 24 operative to cause distal end deflection 22 from a straight condition of shaft 12, as shown in dashed-line format, to a deflected condition, as shown in solid line.

A controller 26 is disposed in a location remote from distal end portion 14 and is configured to produce actuation input 24, which is communicated to actuator 20 without altering a mechanical characteristic or property of the remainder portion 16 of shaft 12. Controller 26 may further include a capability of rotating shaft 12, for example, in the direction of double arrow-headed line 28.

Remote actuator 20 may comprise conventional apparatus known in the art, and may comprise one or more of a magnetic actuator, a material phase change-based actuator, a hydraulic actuator, a piezo-electric actuator, an electric actuator or a combination of any one or more of the foregoing actuator types. A magnetic actuator may include permanent magnets, electro-magnets, or solenoids. One of ordinary skill in the art would appreciate that size, weight and/or heat are appropriate criteria to consider in configuring any particular embodiment. With respect to a material phase change-based actuator, such an actuator may comprise thermally induced force/displacement structures, for example, using shape memory alloy (SMA) materials (e.g., nickel titanium alloys, such as Nitinol material (NiTi) wire coils or wire forms). It should be further appreciated that useful force and displacement outputs from appropriate configuration size, phase change-based coils or wire forms may be achieved. In the case where remote actuator 20 comprises a material phase change-based actuator, such actuator 20 may include at least one coil or wire form comprising SMA material configured to transition, responsive to actuation input 24, from a first state to a second state where the second state has a different physical or mechanical configuration as compared to the first state (e.g., an expanded length versus a shortened or contracted length). With respect to a hydraulic actuator, such a remote actuator may operate based on pressurized fluid, such as saline solution.

Controller 26 may comprise, in some embodiments, a manually-actuated “hand” controller, depending upon the nature of remote actuator 20 and the corresponding actuation input 24. In further embodiments, controller 26 may comprise an electronic controller configured to appropriately generate an electric or electronic actuation input signal 24. In still further embodiments, controller 26 may comprise a hydraulic controller configured to control a fluid pressure, which operates as the actuation input 24.

In still further embodiments, controller 26 may configured to vary the actuation input 24 in accordance with changes in one or more of a measured or estimated condition or parameter, which may be internal a catheter in which the remote actuator is deployed or external thereto. Remote actuator 20, in turn, is responsive to the varying actuation input 24 to thereby vary the controlled movement or response. Such embodiments may be useful, for example, in navigation of the catheter (or distal tip thereof) where the monitored condition or parameter is the tip position (or position and orientation). One of ordinary skill in the art will appreciate that a wide variety of feedback-controlled applications are possible.

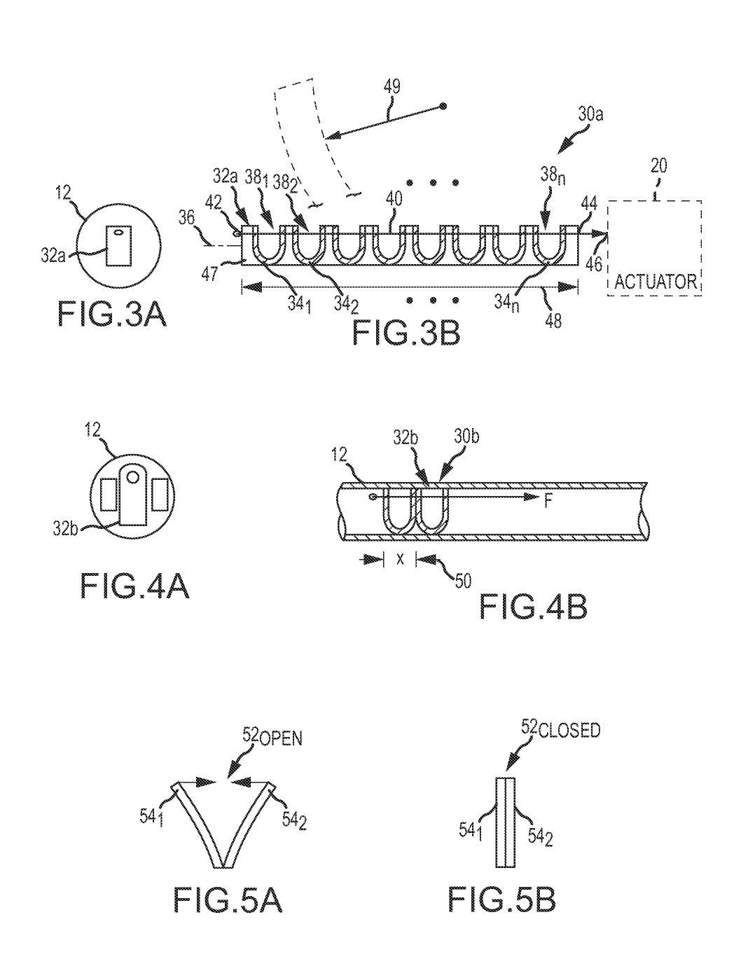

FIGS. 3A-3B are end and side (cross-sectional) views of a first deflection mechanism 30a configured for use with remote actuator 20. Mechanism 30a is responsive to the controlled movement delivered by actuator 20 to achieve deflection, for example, distal end deflection in a catheter. FIG. 3A shows a spring assembly 32a included in deflection mechanism 30a housed within an outer catheter body or shaft 12. When spring assembly 32a deflects, shaft 12 also deflects therewith.

FIG. 3B shows deflection mechanism 30a in greater detail. Spring assembly 32a comprises a plurality of springs 34₁, 34₂, . . . 34ₙ extending along an axis 36. Each spring 34₁, 34₂, . . . 34ₙ has a respective open end 38₁, 38₂, . . . 38ₙ. Mechanism 30a further includes an elongate member 40 having a first end 42 fixed at a first axial end of spring assembly 32a. Elongate member 40, as illustrated, passes through springs 34₁, 34₂, . . . 34ₙ proximate open ends 38₁, 38₂, . . . 38ₙ and extends out of a second axial end 44 of spring assembly 32a. The second end 46 of member 40 is coupled to remote actuator 20. Member 40 may be a wire, a thread or other structure capable of transmitting a force. The plurality of springs 34₁, 34₂, . . . 34ₙ, may be embedded in a base 47 comprising relatively flexible material (e.g., polymer).

Actuator 20 is configured to impart a controlled movement (i.e., as shown in the direction of the arrow head in FIG. 3B) to member 40, thereby causing spring assembly 32a to deflect. The deflection imparted by mechanism 30a is shown in dashed-line format in FIG. 3B. When the actuation signal supplied to actuator 20 is removed or otherwise de-asserted by controller 26, member 40 is taken out of tension and a resilient, return force exhibited by springs 34₁, 34₂, . . . 34ₙ is no longer counteracted by the force applied by member 40. The return force exhibited by the springs 34₁, 34₂, . . . 34ₙ is sufficient to overcome (deform) the shaft material away from the deflected position back to being straight (i.e., the springs 34₁, 34₂, . . . 34ₙ, and the outer shaft return to being straight). FIG. 3B further shows spring assembly 32a having a nominal axial length 48, which in turn determines a bend radius 49 associated with mechanism 30a.

FIGS. 4A-4B are end and side (cross-sectional) views of a second embodiment of a deflection mechanism, designated mechanism 30b. Mechanism 30b is also configured for use with remote actuator 20, and is likewise responsive to the controlled movement delivered by actuator 20 to achieve distal end portion deflection. FIG. 4A shows a spring assembly 32b that is included in deflection mechanism 30b, and which is housed within an outer catheter body or shaft 12. When spring assembly 32b deflects, shaft 12 also deflects therewith in a corresponding manner.

FIG. 4B shows deflection mechanism 30b in greater detail. As with deflection mechanism 30a, deflection mechanism 30b includes a plurality of substantially U-shaped springs 34₁, 34₂, . . . 34ₙ. Each spring 34₁, 34₂, . . . 34ₙ has a nominal axial width 50, designated x in FIG. 4B, which in addition to the overall length of spring assembly (see length 48 in FIG. 3B) also influences the bend radius. The width 50 associated with springs 34₁, 34₂, . . . 34ₙ also influences the uniformity of the bend radius provided by deflection mechanism 30b. For example, each one of the springs 34₁, 34₂, . . . 34ₙ may have the same width x, which provides for a uniform bend radius when operated. However, the respective widths x for each one of the springs 34₁, 34₂, . . . 34ₙ may be adjusted in magnitude, uniformly for all springs, as well as varied from spring to spring so as to provide both greater or lesser amounts of overall deflection as well as to provide regular (uniform) or irregular bend radii. Spring assembly 30b may thus be configured, through variation in respective spring widths 50 as well as in the overall spring assembly length 48, to provide a predetermined, desired bend profile (i.e., size, regular or irregular bend radii, etc.). Moreover, one of ordinary skill in the art will appreciate that in further embodiments of spring assemblies 32a, 32b, the geometry and/or shape of the individual springs 34₁, 34₂, . . . 34ₙ may also be varied so as to achieve still further customization of the deflection amount and bend radii.

FIGS. 5A-5B are respective side views of a V-shaped spring 52 having constituent side portions 54₁ and 54₂. Spring assembly 32a, 32b, in further embodiments, may include a substantially V-shaped spring 52 instead of a substantially U-shaped spring. In this regard, FIG. 5A shows spring 52 in an open condition, designated 52ₒₚₑₙ while FIG. 5B shows spring 52 in a closed condition, designated 52_{closed}. A V-shaped spring 52 provides the capability of providing a tighter radius than a U-shaped spring, as in FIGS. 3A-3B and FIGS. 4A-4B.

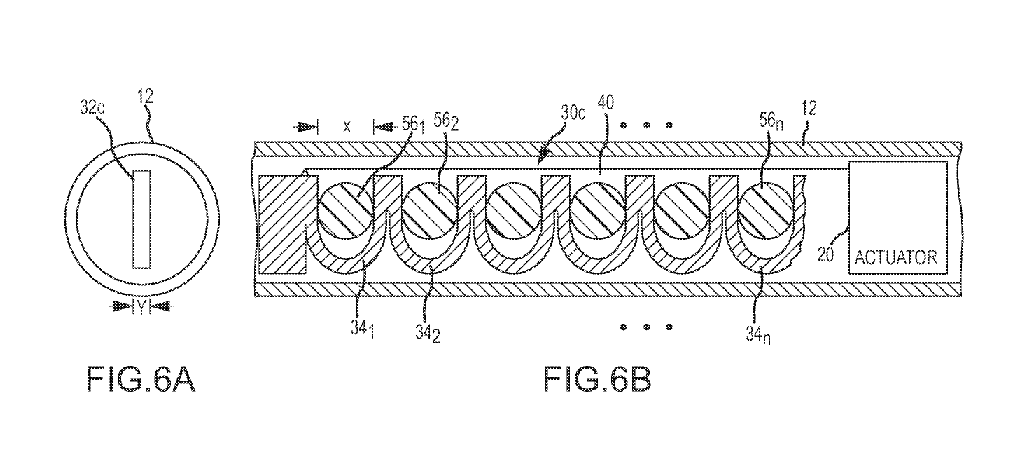

FIGS. 6A-6B show a further embodiment of a deflection mechanism, designated mechanism 30c. Deflection mechanism 30c contains a spring assembly 32c, which as shown in FIG. 6A has a nominal width in the radial direction designated Y. Spring assembly 32c is substantially the same as spring assemblies 32a and 32b with the exception that assembly 32c further includes a plurality of resilient return members 56₁, 56₂, . . . 56ₙ disposed in respective recesses of springs 34₁, 34₂, . . . 34ₙ. Each spring 34₁, 34₂, . . . 34ₙ includes a base level of resiliency operative to return the spring assembly to its original configuration (e.g., straight) when the force applied by member 40 is released. However, resilient return members 56₁, 56₂, . . . 56ₙ increase the effective resiliency, thereby increasing the total return force output by spring assembly 32c.

The resilient return members 56₁, 56₂, . . . 56ₙ, being disposed in the spring recesses, are compressed when spring assembly 32c is deflected by actuator 20. When deflected, the total return force that actuator 20 must overcome, ignoring for the moment any additional force required to deflect other materials, such as the outer shaft, corresponds to the sum of (i) the return force contributed by springs 34₁, 34₂, . . . 34ₙ and (ii) the return force contributed by compressed return members 56₁, 56₂, . . . 56ₙ. When actuator 20 releases member 40, the composite return force operates to return spring assembly 32c to an original, non-deflected state.

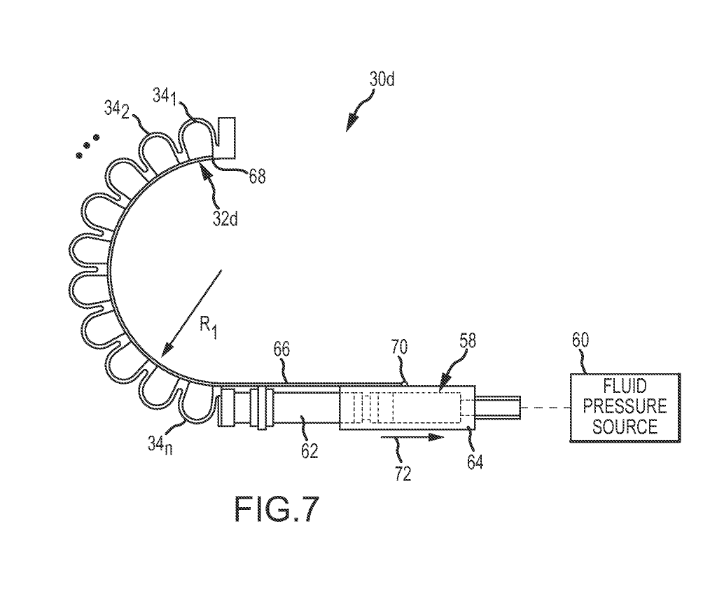

FIG. 7 is a simplified, side view of a further deflection mechanism, designated mechanism 30d. Deflection mechanism 30d includes a spring assembly 32d that includes a plurality of individual springs (e.g., U-shaped as illustrated) designated 34₁, 34₂, . . . 34ₙ. Deflection mechanism 30d is coupled to a hydraulically-actuated cylinder 58 driven by with a fluid pressure source 60 (e.g., where the source of pressure may be saline fluid). Hydraulic actuator 58 includes a piston assembly 62 and an outer sleeve 64, all shown in simplified fashion in FIG. 7. An interior of sleeve 64 forms a chamber into which pressurized fluid is selectively introduced. The piston 62 and sleeve 64 are configured to move, one relative to the other. As shown, one end of piston assembly is connected to spring assembly 32d at spring 34ₙ.

Deflection mechanism 30d further includes an elongate member 66, which passes through the plurality of individual springs 34₁, 34₂, . . . 34ₙ and is affixed to distal spring 34₁, for example at point 68. The axially opposite end of elongate member 66 is coupled to sleeve 64, for example at point 70. Elongate member 66 passes through the open end of the remainder of springs 34 (i.e., member 66 need not be fixed to each of the intervening springs).

In operation, fluid pressure in the fluid chamber of hydraulic actuator 58 (controllably provided by source 60) operates against piston 62 to develop a force, as known, based in part on the area of the piston and the pressure of the fluid. The developed force tends to move piston assembly 62 and sleeve 64 apart. Inasmuch as the piston assembly 62 is “grounded” to the proximal end of the spring assembly 32d, movable sleeve 64 will move in a direction designated by arrow 72. It should be understood that the force developed by actuator 58 must exceed a force needed to compress spring assembly 32d in order to achieve movement of the sleeve 64. As a result of the movement of sleeve 64, elongate member 66 is placed in tension, spring assembly 32d compresses the deflection mechanism 30d deflects in a manner illustratively shown in FIG. 7. The degree of deflection may be controlled by varying the pressure of the fluid provided by fluid pressure source 60. The spring assembly 32d is shown having a bend radius of R₁, although in light of the foregoing description it should be understood that lesser or greater degrees of curvature are possible, depending on the applied fluid pressure. In addition, it should be further understood that spring assembly 32d may be varied, for example, at least as described above in connection with spring assemblies 32a, 32b and 32c.

Fluid pressure source 60 may be controlled to reduce the applied fluid pressure to hydraulic actuator 58, with the result that the restorative force exhibited by the individual springs of spring assembly 32d will urge the assembly 30d away from its then-deflected state towards its original, un-deflected state. Degrees of reduction in fluid pressure will result in corresponding reductions in the degree of curvature (or bend radius). Upon a sufficient reduction in or a total removal of fluid pressure being delivered by fluid pressure source 60, the resilient, return force exerted through the springs of spring assembly 32d returns assembly 30d back to its original, non-deflected state.

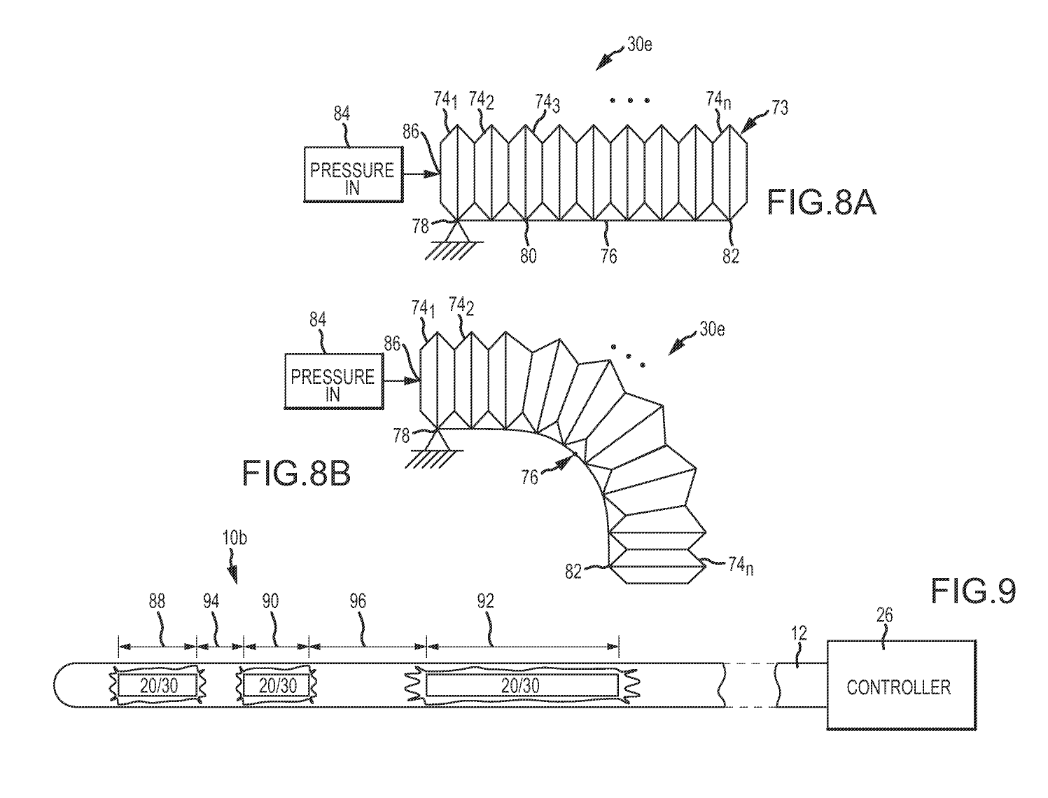

FIGS. 8A-8B are diagrammatic, side views of a further embodiment of a deflection mechanism, designated deflection mechanism 30e. FIG. 8A shows a bellows assembly 73 of deflection mechanism 30e in a first, non-deflected state, while FIG. 8B shows bellows assembly 73 in a pressurized, deflected state. Bellows assembly 73 comprises a plurality of chambers 74₁, 74₂, . . . 74ₙ extending along an axis in an un-pressured state. Each chamber 74₁, 74₂, . . . 74ₙ is in fluid communication with an adjacent one of the chambers 74₁, 74₂, . . . 74ₙ. Deflection mechanism 30e further includes an elongate member 76 having a first end 78 fixed at a first axial end of bellows assembly 73. The mechanism 30e is configured such that member 76 is passed through or at least coupled with the intervening (non-end) chambers 74₂, . . . 74ₙ₋₁, for example, as shown at point 80. The coupling of member 76 to the chambers 74 may occur at a transverse side (i.e. top or bottom as per the orientation shown in FIG. 8A). The member 76 further includes a second end 82 that is fixed at a second axial end of bellows assembly 73, as also shown. The member 76 is configured to constrain expansion of one transverse side of bellows assembly 73 while allowing the opposing, unconstrained transverse side to expand. As shown in FIG. 8A, a source of pressurized fluid 84 (e.g., saline solution) is in fluid communication with an inlet 86 of bellows assembly 73 at the first axial end thereof.

FIG. 8B shows mechanism 30e in a deflected state. The source of pressurized fluid 84 may be controlled (e.g., by a controller 26—not shown in FIGS. 8A-8B) to deliver fluid to fill bellows assembly 73. Thereafter, increases in the fluid pressure will cause the chambers 74₁, 74₂, . . . 74ₙ to expand. As described above, since the lower transverse side is constrained from expanding by virtue of member 76, while the upper transverse side is not, the mismatch in the resulting lengths of the two transverse sides results in mechanism 30e deflecting, characterized by a predetermined bend radius, as shown. In the embodiment of FIGS. 8A-8B, the actuator and deflection mechanism are combined, with the actuation input comprising the pressurized fluid that is being delivered to bellows assembly 73. It should be appreciated that delivery of pressurized fluid through a catheter (e.g., in a fluid lumen) will not substantially alter the mechanical characteristics of the catheter body.

FIG. 9 is a diagrammatic view of a further catheter embodiment, designated catheter 10b. Catheter 10b includes a plurality of remote actuator/deflection mechanism combinations, three of which are shown (each designated by reference numerals “20/30”). As described above, each remote actuator 20 and deflection mechanism 30 may have its own unique bend radius and/or shape (e.g., irregular bend radii). For example, where the deflection mechanism is a spring assembly, the bend radius is defined not only its overall length, but also by the respective lengths of each individual spring 34 in the assembly, and whether any particular spring length has been varied relative to other individual spring lengths.

In view of the foregoing, catheter 10b includes a plurality of actuator/deflection mechanism combinations, such as a first actuator/deflection mechanism combination having an overall length 88, a second actuator/deflection mechanism combination having an overall length 90, and a third actuator/deflection mechanism combination having an overall length 92. In addition, the spacing between actuator/deflection mechanism combinations may be the same or may be different, as illustrated in FIG. 9. For example, a first spacing 94 may be different than a second spacing 96. Through variation in the individual lengths of the actuator/deflection mechanism, along with variation in the spacing between such combinations, a predetermined, desired curvature may be achieved.

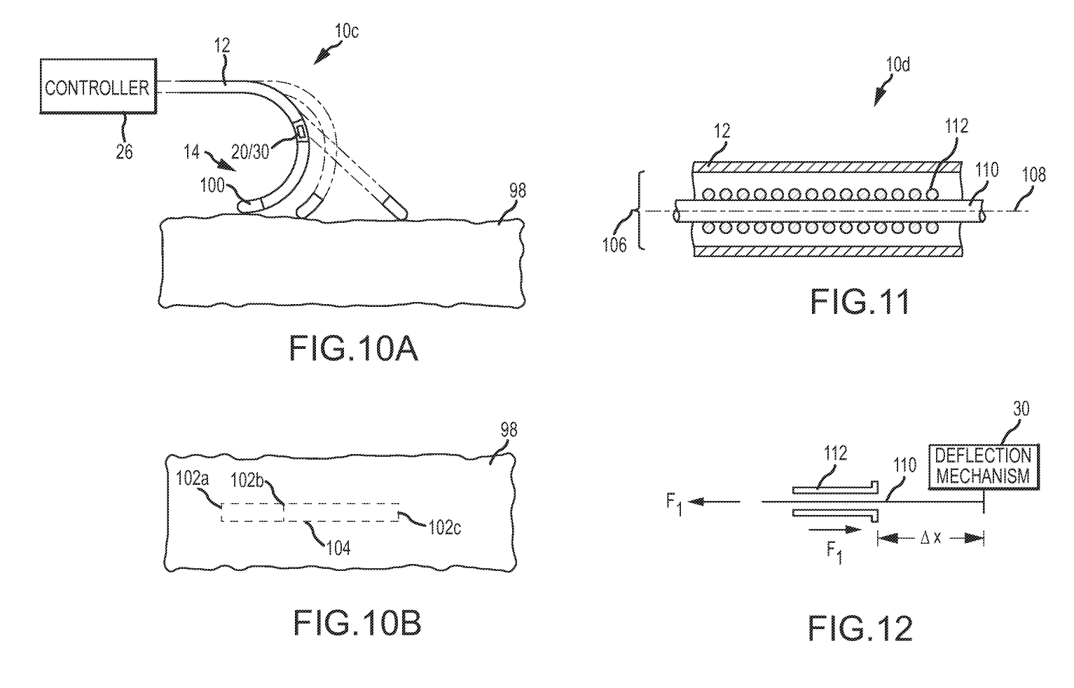

FIGS. 10A-10B are diagrammatic, side and top views, respectively, of a further catheter embodiment, designated catheter 10c. Catheter 10c includes a remote actuator/deflection capability, which may be used in connection with a wide variety of diagnostic and/or therapeutic procedures. FIGS. 10A-10B illustrate such a capability as used in an exemplary electrophysiological (EP) procedure, namely, an ablation procedure for creating a linear lesion.

FIG. 10A shows tissue 98, which may be cardiac tissue that has been previously determined (e.g., through other procedures) to have a target volume of tissue, which will be the target of an ablation procedure. Catheter 10c, as illustrated, includes shaft 12 having a proximal end portion and distal end portion 14 and wherein the proximal end portion is coupled to controller 26. The catheter 10c also includes a remote actuator 20 deployed in combination with a deflection mechanism 30, such combination being designated 20/30 in the Figure. The remote actuator/deflection combination 20/30 is deployed at the distal end portion 14. In addition, an ablation tip 100 is disposed at an extreme distal end of shaft 12. Ablation tip 100 may comprise conventional materials and configurations. It should be understood that many components, elements and features of an ablation system have been omitted for clarity.

The procedure for creating a linear lesion using ablation first involves causing the distal end portion 14 of catheter 10c to be deflected through activation of actuator 20. As described above, the activation of actuator 20 may in turn involve controller 26 producing an actuation input 24 (not shown) that is provided to actuator 20. Depending on the type of actuator used, a plurality of different actuation inputs may be used (e.g., electric, hydraulic, etc.). The catheter 10c is also navigated into to the target site. It should be appreciated that communicating the actuation input does not alter the mechanical properties of the main catheter body, as described above. As a result of the foregoing, ablation tip electrode 100 is positioned in contact with tissue 98 substantially at position 102a (best shown in FIG. 10B).

The procedure next involves applying ablative energy (e.g., radio-frequency (RF) ablation energy) to tissue 98 via the ablation tip electrode 100. It should be understood that other ablative energy modalities may be used and depending on the ablative energy type, a different ablation tip 100 may be used.

The procedure next involves progressively relaxing the degree of deflection of the distal end portion 14 of shaft 14, which allows the deflection mechanism 30 to return to its original, non-deflected state. As shown in dashed-line format in FIG. 10A, as the relaxation occurs, the ablation tip electrode 100 sweeps forward as the catheter distal end portion 14 returns to an original, non-deflected state. In reference to FIG. 10B, ablation tip electrode 100 travels through locations 102a, 102b, and ends its travel at location 102c. The area (volume) of tissue that is ablated (“linear lesion”) is enclosed in a dashed-line box in FIG. 10B. This step may be performed substantially simultaneously with the previous step (i.e., applying ablative energy). In an embodiment, controller 26 performs this step by progressively discontinuing the actuation input that is communicated to actuator 20.

FIG. 11 is a partial, cross-sectional view of a catheter 10d showing a coaxially extending pull wire arrangement. The arrangement requires a reduced force as compared to the conventional pull wire arrangements (i.e., pull wires that are offset from the main axis of the catheter body, or are embedded in the shaft wall). The arrangement in FIG. 11, like the remote actuator embodiments described above, are particularly useful in the remote activation of distal end functions on a catheter without altering the mechanical characteristics of the main catheter body.

In FIG. 11, a further catheter embodiment, designated catheter 10d, includes a radially outermost shaft 12, and an actuation mechanism 106 that extends along a main axis 108 of catheter 10d. The mechanism 106 may include a centrally-disposed pull wire 110, which is arranged in a coaxial fashion with respect to the main axis 108. The mechanism 106 may further include a flexible sleeve 112, which may comprise a coil, a hypo tube or the like, and that is configured to provide a buttress for an opposing force to that force that is applied to the pull wire 110, as more fully explained below.

FIG. 12 is a diagrammatic and block diagram of the mechanism of FIG. 11. As shown, a “pull” wire force of F₁ that is applied to central pull wire 110 may be counter-acted by an equal and opposite force F₁ that is applied to flexible sleeve 112, which, for example, may extend to the proximal end of catheter 10d and at which end the counter-acting force may be applied. In some embodiments, the proximal end of the sleeve 112 is “grounded” for example, in a handle (i.e., a manually-actuated controller 26) or the like so as to be able to counteract the pull wire force. Through the foregoing mechanism, the pull force and the reaction force are constrained to exist only in elements 110 and 112, respectively, thereby maintaining the other components of catheter 10d, for example shaft 12, in a substantially force free state. This mechanism enables the deployment of a distally-located deflection mechanism 30, which as shown in FIG. 12 may be configured to be responsive to an actuation input provided in the form of a force transmitted through pull wire 110 (i.e., force F₁). The resulting force on pull wire 110 thus acts on or is otherwise provided to deflection mechanism 30. In sum, the foregoing mechanism results in no net effect on the catheter body.

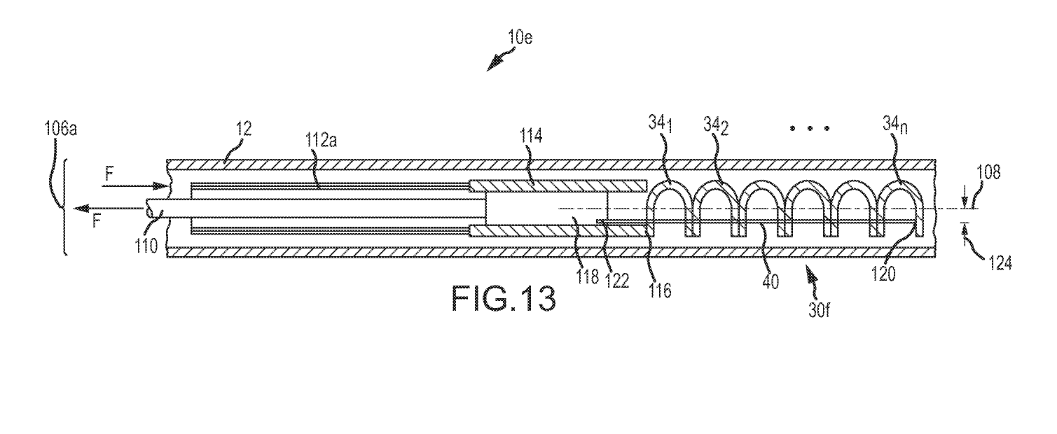

FIG. 13 is a side, partial, cross-sectional view of a further catheter embodiment, designated catheter 10e. Catheter 10e combines a coaxially-extending actuation mechanism 106a and a deflection mechanism 30f, similar those described above in connection with FIGS. 3A-3B, for example. The actuation mechanism 106a includes a central pull wire 110 and a flexible, reaction sleeve 112a. Reaction sleeve 112a is coupled to a cylinder 114. The pull force (F) applied to pull wire 110 acts as an actuation signal while the reaction force (also F) that is resolved into reaction sleeve 112a counter-acts the applied pull force, constraining the forces to exist in only those two elements, resulting is substantially no net force existing elsewhere (e.g., no net force in shaft 12).

Deflection mechanism 30f includes a plurality of individual springs 34₁, 34₂, . . 34ₙ. The proximal end 116 of deflection mechanism 30f is fixed to cylinder 114. Central pull wire 110 is coupled to a movable element 118 that is configured to move axially within cylinder 114. Deflection mechanism 30f also includes an elongate member 40, which is fixed at an extreme distal end 120 of mechanism 30f and is further coupled at an extreme proximal end thereof to movable member 118. Elongate member 40 extends generally along a minor axis that is substantially parallel to but offset from main axis 108 by an amount 124. The offset amount 124 is taken in a radial direction.

In operation, a pull force (F) applied to pull wire 110 operates to move movable member 118 relative to cylinder 114. This movement, in turn, places elongate member 40 in tension, where the pull force (F) that is applied across the spring assembly comprising springs 34₁, 34₂, . . . 34ₙ, is resolved at point 116 of cylinder 114. Note, that cylinder 114 is substantially stationary since the reaction force (F) applied to sleeve 112a, and then to cylinder 114, counteracts the pull force (F) which is applied across the springs (via pull wire 110, element 118 and member 40) and then also to cylinder 114. As a result, deflection mechanism 30f, particularly springs 34₁, 34₂, . . . 34ₙ, deflect, as described above. Through the foregoing arrangement, distal end deflection may be achieved without altering the mechanical characteristics of the main body of catheter 10e (e.g., shaft 12).

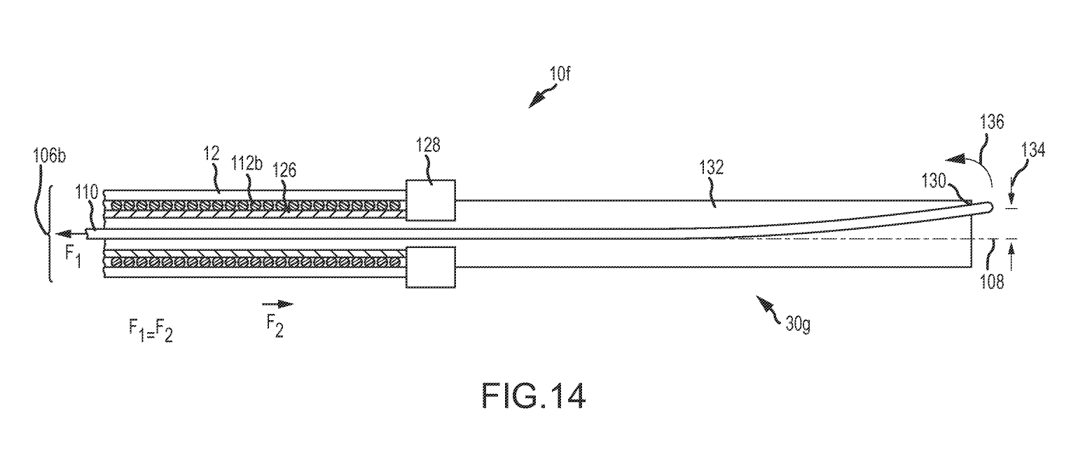

FIG. 14 is a partial, side view, with portions broken away, of a further catheter embodiment, designated catheter 10f. Catheter 10f is similar to catheter 10e, except that an alternate deflection mechanism 30g is employed. Catheter 10f includes an actuation mechanism 106b including a centrally-disposed pull wire 110 (i.e., coaxially-extending with the main axis of the catheter) and a flexible coil 112b that is disposed, in the illustrated embodiment, radially inwardly of shaft 12. FIG. 14 further includes a liner 126 that is shown radially inwardly of coil 112b but which is radially outwardly of central pull wire 110. As with the previous embodiments, a proximally directed “pull” force F₁ is applied to central pull wire 110, wherein a reaction force F₂ is resolved into the base of coil 112b. A distal portion of coil 112b engages a buttress 128.

Deflection mechanism 30g further includes a flexible member 132, which may comprise a compliant beam 132. A proximal end of beam 132 engages buttress 128. In addition, the distal end of pull wire 110 is affixed to beam 132 at a distal point 130 that is transversely (i.e., radially) offset from main axis 108 by an amount designated 134. In operation, application of a relatively low, proximally-directed pull force (F₁) to pull wire 110 causes a deflection of beam 132 in substantially the direction of arrow 136. The pull force applied across beam 132, and which is resolved into buttress 128 is counteracted by the reaction force F₂ that is applied coil 112b, and also resolved into buttress 128.

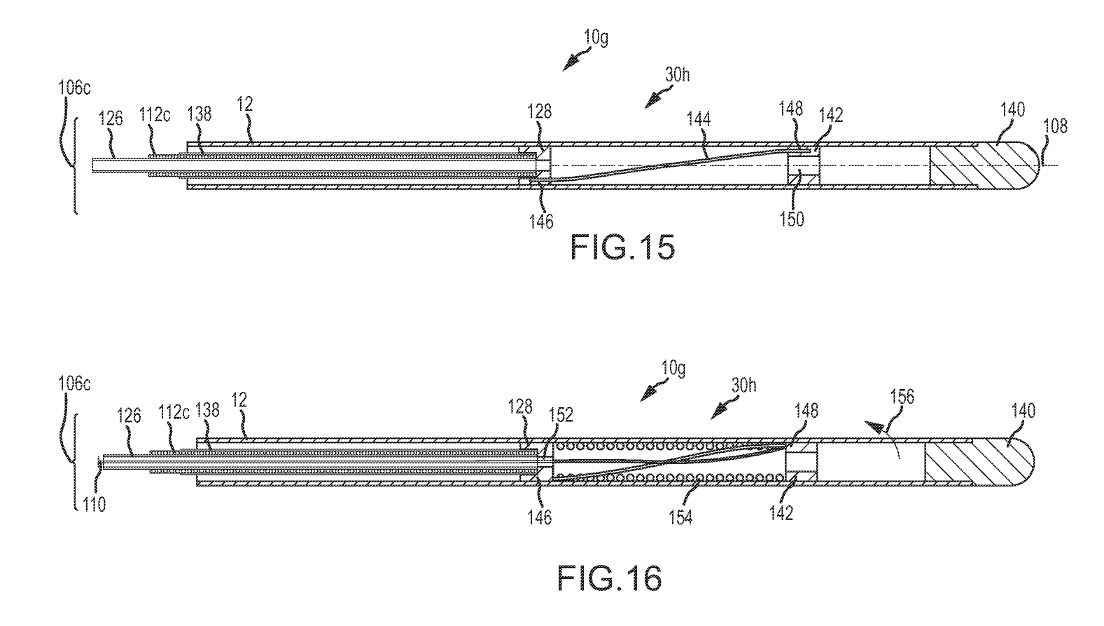

FIGS. 15-16 are partial, side, cross-sectional views of a further catheter embodiment, designated catheter 10g. FIG. 15 shows catheter 10g in a first stage of manufacture while FIG. 16 shows a completely assembled catheter 10g.

In FIG. 15, an actuation mechanism 106c includes a central pull wire 110 (best shown in FIG. 16) and a flexible, reaction coil 112c which, as described in connection with the previous embodiments, may be mechanically “grounded” in a proximally disposed handle controller while a distal end thereof engages buttress 128. Catheter 10g further includes a liner 126, which is shown being disposed radially inwardly of flexible reaction coil 112c, and a cover 138, which is shown being disposed radially outwardly of flexible reaction coil 112c. Catheter 10g further shows, in the illustrated embodiment, a distal electrode 140, although it should be understood that inclusion of electrode 140 is exemplary rather than limiting in nature.

Catheter 10g also includes a further embodiment of deflection mechanism 30, designated deflection mechanism 30h. Deflection mechanism 30h includes a ring 142 disposed axially distal of buttress 128 and a spar member 144. Spar member 144 extends generally axially between buttress 128 and ring 142 and is configured to promote deflection of deflection mechanism 30h in a predetermined plane upon application of an actuation input (i.e., in this embodiment, the pull force applied to pull wire 110), and is further configured to facilitate the return motion of deflection mechanism 30h from the deflection position back to an original, non-deflected (“home”) position upon the removal of the actuation input. As shown in cross-section in FIG. 15, spar member 144, when taken in axial cross-section, is arranged in a diagonal orientation, being coupled on a first side of main axis 108 (e.g., at point 148) and a second end of spar member 144 being coupled to buttress 128 on a second side of axis 108 opposite the first side (i.e., at point 146).

The spar member 144 is configured to deflect from a first state to a second state when a first force is applied to pull wire 110 and wherein spar 144 is further configured to provide a restoring force when the “pull” force on pull wire 110 is discontinued.

FIG. 16 shows a further stage of manufacture of catheter 10g, now further showing pull wire 110 and a spring 154. An extreme distal end of pull wire 110 is coupled to ring 142 at a location near spar member 144. Note, that the connection point of the distal end of pull wire 110 is offset from main axis 108, just like that in the embodiment of FIG. 14. Buttress 128 includes a centrally disposed through-bore configured to allow pull wire 110 to pass therethrough. Deflection mechanism 30h also includes spring 154, which provides stability between buttress 128 and ring 142, thereby relaxing the requirements for the selection of material for outer shaft 12. That is, the spring 154 becomes the main influence in determining the magnitude of the bend radius that results from a particular pull force as well as the uniformity of the bend itself. A straight spring 154, as shown, will result in a substantially uniform bend radius, while the degree of deflection that will occur for any particular pull force will depend on the spring constant and spring radius (geometry), as well as material properties of the shaft, to a lesser extent. When a “pull” force (i.e., the actuation input) is applied to pull wire 110, the distal end portion of catheter 10g deflects in a direction substantially indicated by single arrow headed line 156.

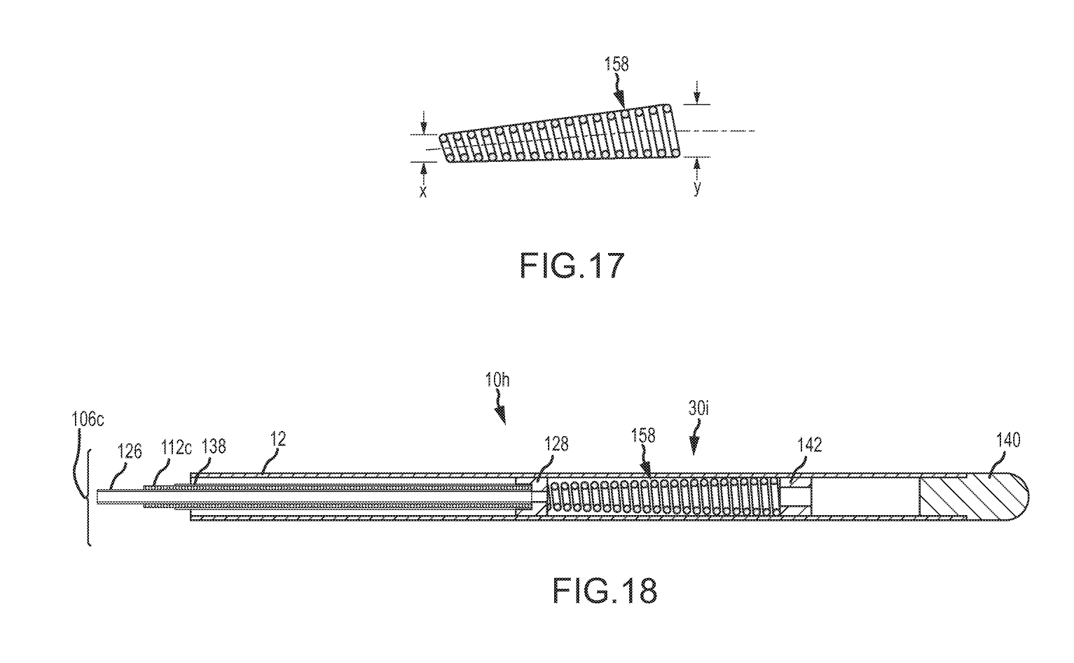

FIG. 17 shows a tapered spring 158 having a maximal diameter of Y and a minimum diameter of X. The use of a tapered spring 158 fulfils some of the same objectives as straight spring 154 (i.e., relaxes material choice selections for the shaft), however, a tapered spring 158 provides the ability to produce non-uniform deflections, inasmuch as the force needed to overcome (and thus bend) the tapered spring 158 at any particular position along its axis is determined as a function of axial position.

FIG. 18 is a partial side, cross-sectional view of a further catheter embodiment, designated catheter 10h. Catheter 10h is substantially the same as catheter 10g, except that catheter 10h includes an alternative deflection mechanism, designated mechanism 30i, which in turn is substantially the same as deflection mechanism 30h of FIGS. 15-16, except deflection mechanism 30i includes tapered spring 158, rather than straight spring 154. It should be understood that in FIG. 18, various components already described above in connection with FIG. 16 have been omitted to more clearly show tapered spring 158. Spring 158 provides for a customized bend radius, as described above in connection with FIG. 18.

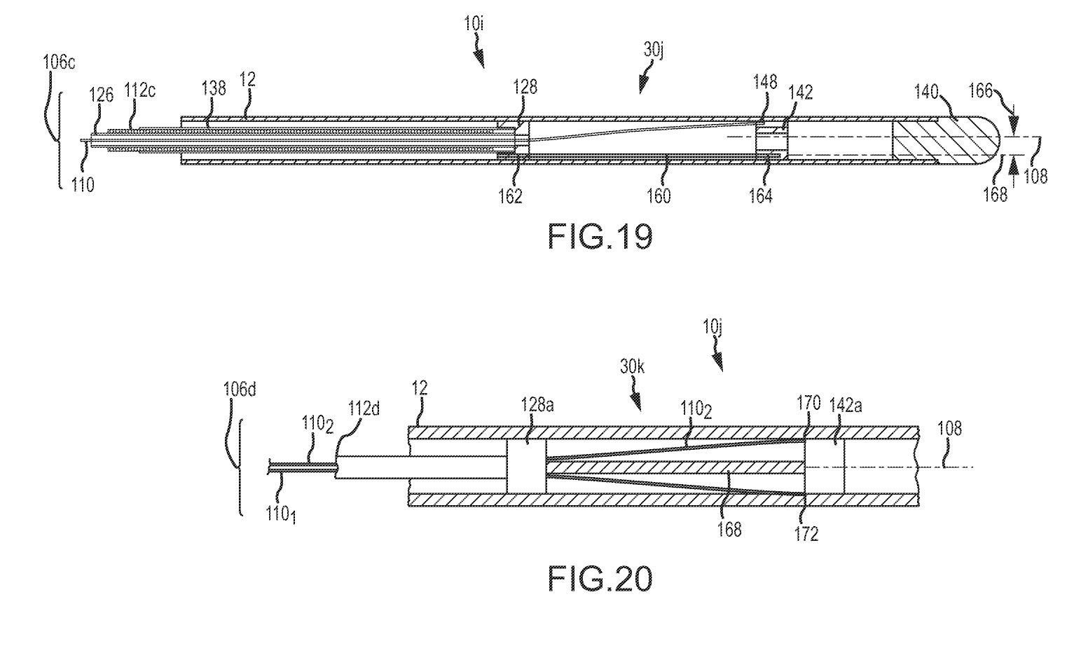

FIG. 19 is a partial, side, cross-sectional view of a further catheter embodiment, designated catheter 10i. Catheter 10i is substantially the same as catheter lOg illustrated in FIG. 16 (and corresponding description), except that catheter 10i includes a further deflection mechanism designated mechanism 30j. Deflection mechanism 30j includes a horizontally extending spar member 160 that is coupled to buttress 128 at point 162 and is further coupled to ring 142 at point 164. Spar member 160 is offset from main axis 108 by an amount 166 relative to axis 168. Spar member 160 controls deflection of the distal end of catheter 10i in the same general manner as described above but also provides a restorative force when the actuation input (i.e., the pull force) is discontinued.

FIG. 20 is a partial, side and cross-sectional view of a further catheter embodiment, designated catheter 10j. Catheter 10j is similar to aspects of catheter 10f of FIG. 14 and catheter 10i of FIG. 19, except that catheter 10j includes a further actuation mechanism 106d and a further deflection mechanism, designated mechanism 30k. Deflection mechanism 30k is responsive to an input from actuation mechanism 106d. Mechanism 106d includes a pair of pull wires 110₁ and 110₂ and a reaction element 112d, which may extend to the proximal end and be “grounded”, like reaction sleeves 112a, 112b, and 112c. The deflection mechanism 30k is responsive to respective pull force transmitted by two pull wires 110₁ and 110₂ to achieve bidirectional deflection. Deflection mechanism 30k includes a modified buttress 128a and a modified ring 142a, as well as a central spar or beam member 168 substantially coaxially arranged with main catheter axis 108 (i.e., beam member 168 extends on an axis coincident with axis 108). A first one of the pull wires, for example pull wire 110₁, may be coupled to a radially-outermost portion of ring 142a, for example at point 172. A second one of the pull wires, for example pull wire 110₂, may be coupled to a radially-outermost portion of ring 142a, for example, at point 170, which is on a side that is opposite that to which first pull wire 110₁ is coupled. Central beam 168 provides a restorative force to return the catheter distal end portion to a straight, non-deflected state when the actuation inputs (pull forces) are discontinued from pull wires 110₁ and 110₂.

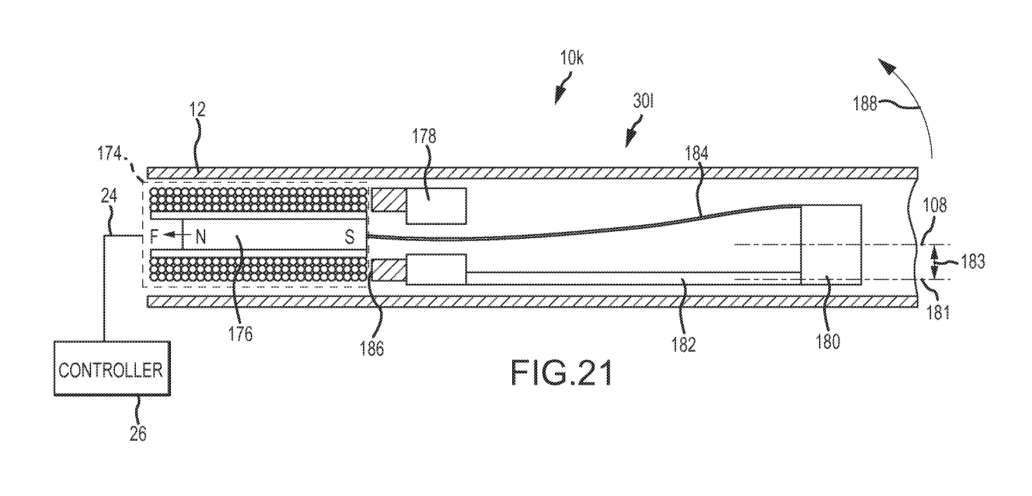

FIG. 21 is a partial, side, and cross-sectional view of a further catheter embodiment, designated catheter 10k. Catheter 10k includes a further deflection mechanism designated mechanism 30l (t-h-i-r-t-y e-l-l). The deflection mechanism 30l comprises a buttress 178, a ring 180, and a spar member 182 extending longitudinally between buttress 178 and ring 180. Spar member 182 is similar to spar member 160 in FIG. 19, in structure and function, and extends longitudinally along an axis 181 that is substantially parallel to but radially offset from main axis 108 by a radial distance 183.

Catheter 10k further includes an actuator in the form of an electric solenoid 174 that is responsive to an actuation input 24 originating with controller 26. Solenoid 174 may comprise conventional components known in the art and may include, among other things, a movable slug 176 whose controlled movement is used to produce deflection. The deflection mechanism 30l further includes a force transmitting member 184, which may take the form of a wire or the like. Wire 184 is coupled on a proximal end thereof to movable slug 176 and on a distal end thereof to ring 180, where it is coupled to a point on ring 180 that is transversely offset relative to main axis 108. As also shown, the point on ring 180 to which the distal end of wire 184 is connected is offset in a radial direction that is opposite the direction in which spar member 182 is radially offset.

In operation, when controller 26 generates actuation input 24, a coil portion of solenoid 174 is energized, thereby creating a force (i.e., a force (F) pointed in the direction of the arrow in FIG. 21) acting on slug 176 urging slug 176 to move in a generally proximally direction. This movement of slug 176 places a wire 184 in tension, thereby transmitting force (F) to ring 180, which in turn causes a deflection in the deflection mechanism 30l in a direction indicated by the single arrow headed line 188. The pull force transmitted through member 184 may be resolved at point 186 of buttress 186. The solenoid 174 counteracts such force. The forces are constrained to exist in only the actuation mechanism and the deflection mechanism. Accordingly, the actuation input (from controller 26) does not substantially alter any mechanical or physical characteristics of the main catheter body (shaft).

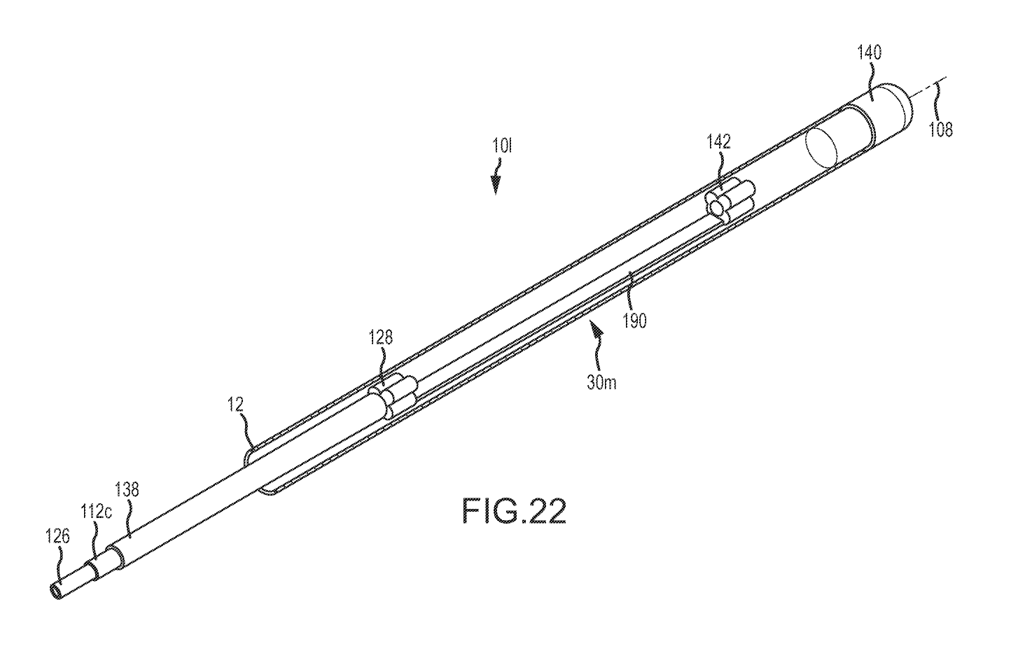

FIG. 22 is an isometric view of a further catheter embodiment, designated catheter 10l (t-e-n e-l-l). Catheter 10l is similar to catheter 10i illustrated in FIG. 19 except that offset beam 190 has replaced spar member 160. In addition, catheter 10l includes an alternative deflection mechanism 30m, which is axially adjustable. In other words, the mechanism 30m may be moved along the axis to change the center of the bend radius. It should be understood that components (e.g., pull wire) have been omitted for clarity.

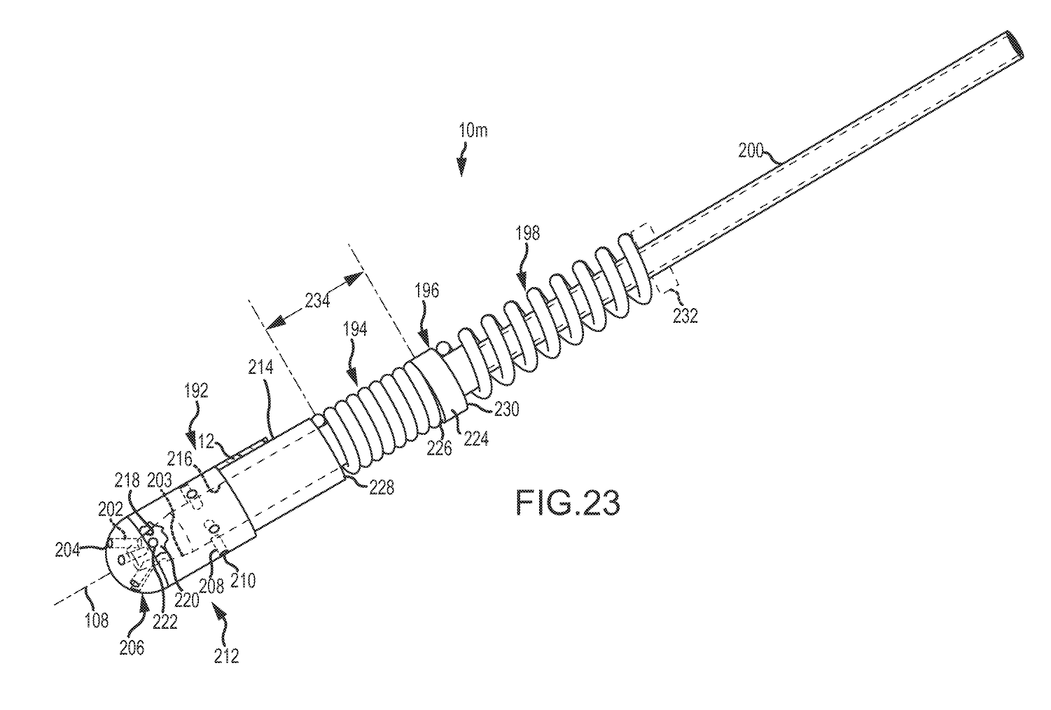

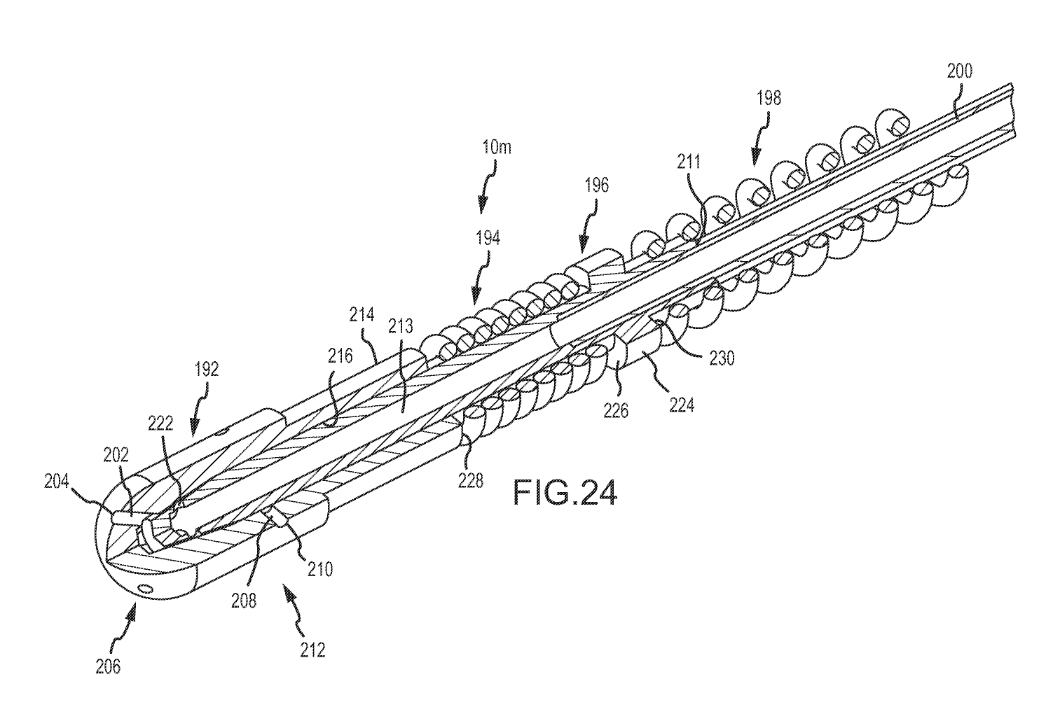

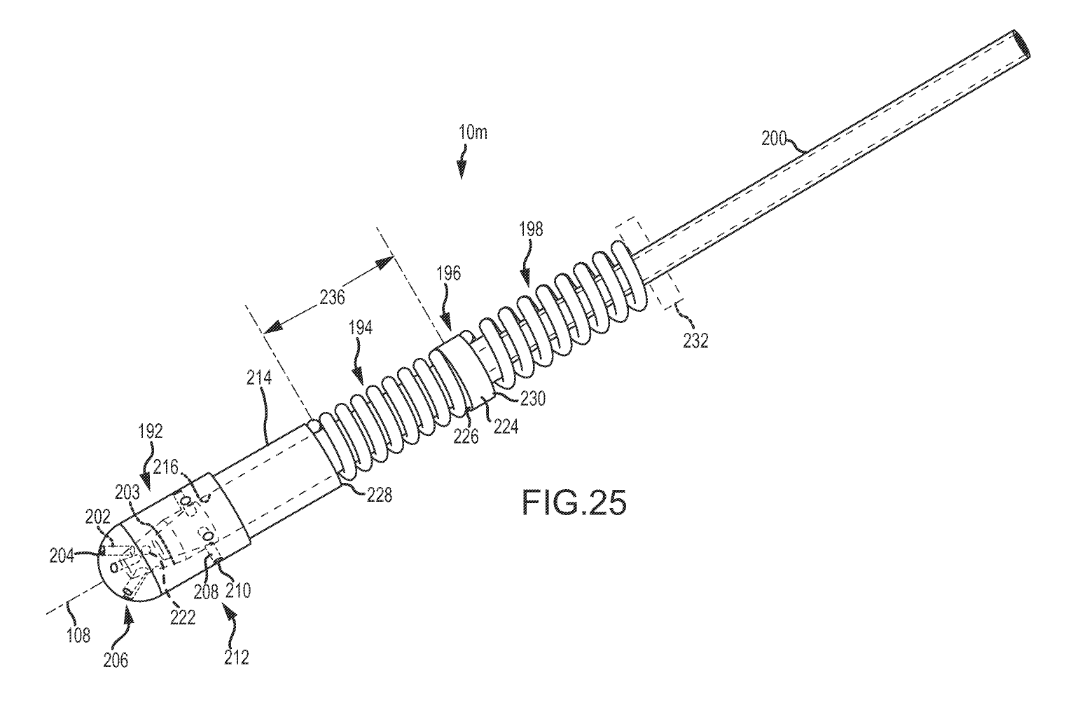

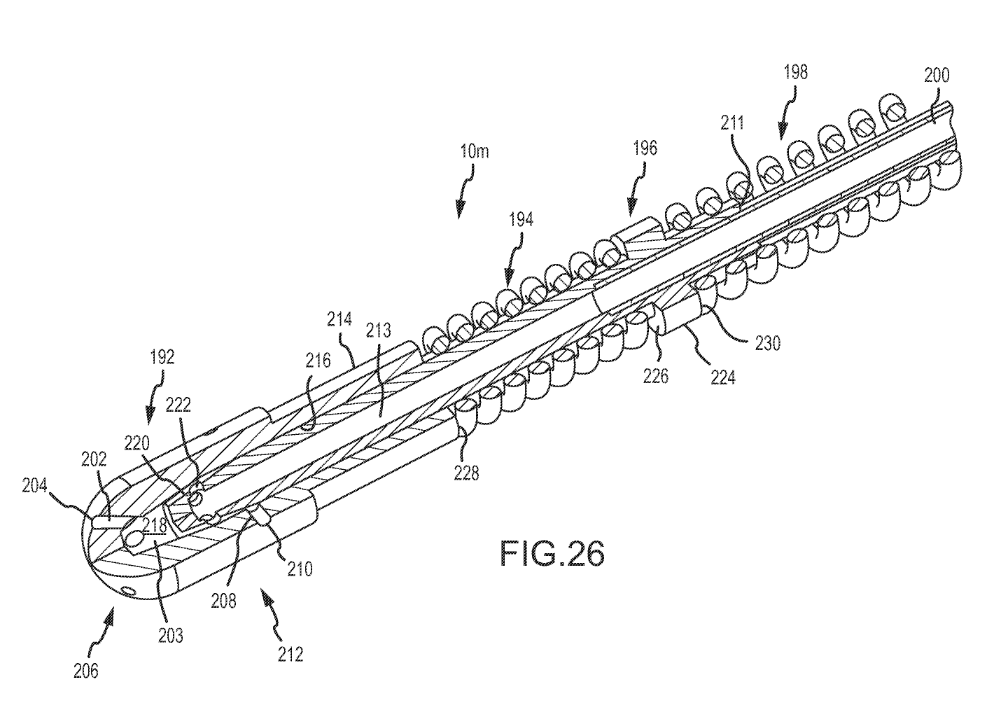

FIGS. 23-26 are isometric views of a further catheter embodiment, designated catheter 10m, in respective closed (FIGS. 23-24) and open (FIGS. 25-26) positions. The catheter 10m illustrated in FIGS. 23-26 illustrate a valve system that is configured to function remotely to provide volume and/or directional control of irrigation fluid to cool tissue near an ablation site and/or to deliver therapeutic agents through a catheter-based system. Closed or open loop feedback mechanisms (not shown) may be included to meter delivery rates and direction. As described below, fluid delivery direction may be controlled by controlling what irrigation ports are opened (i.e., whether distal irrigation ports are opened, whether proximal irrigation ports are opened or whether both are opened).

Catheter 10m includes a distal ablation electrode 192, an actuator coil or wire form 194, a valve spool 196, a counter coil or wire form 198 and an irrigation fluid delivery tube 200.

Ablation electrode 192 includes proximal, generally cylindrical shaped shank 214 and a distal, main body portion having a generally convex (e.g., hemispherical as shown) surface 206 configured for tissue ablation. The proximal shank 214 is configured in size and shape to receive thereon a conventional catheter shaft 12, shown substantially broken away in FIG. 23. Electrode 192 further includes a plurality of distal irrigation passageways 202 extending from a fluid distribution manifold 203, shown in dashed-line format in FIG. 23 (best shown in the cross-sectional view of FIG. 26). The passageways 202 terminate in respective irrigation ports or openings 204 at surface 206. Ablation electrode 192 may further include a plurality of proximal irrigation passageways 208, also extending from manifold 203 and terminating in a corresponding plurality of irrigation ports or openings 210 at a proximal portion 212 of electrode 192. In addition, electrode 192 includes a centrally-disposed proximal opening that leads to a cylindrical shaped bore 216. Various portions of the fluid transport path (e.g., manifold, passageways, etc.) may be insulated. Except as otherwise described herein, ablation electrode 192 may comprise conventional materials and may be constructed using known approaches.

Actuator coil or wire form 194, in concert with counter-coil 198, is configured to impart a controlled movement to valve spool 196, for opening and closing the fluid valve (i.e., selectively exposing or concealing irrigation ports). The actuator coil 194 may comprise, for example, a shape memory alloy (SMA), such as a Nitinol (NiTi) coil with thermal recovery properties. Counter coil 198 may comprise a conventional coil spring, although counter coil 198 may be comprise a Nitinol coil or wire form similar to coil 194, rather than a conventional coil spring. Of course, variations are possible. For example, the entire actuator function may be achieved using solenoids, piezo-electric type, or other actuation methodologies as described herein.

Valve spool 196 includes a fluid inlet 211 (best shown in FIG. 24) located at the proximal end thereof configured to receive fluid delivery tube 200. Inlet 211 is configured to sealingly engage fluid delivery tube 200. Valve spool 196 is internally configured with an irrigation channel 213 extending internally in valve spool 196 from the proximal inlet 211 to a distal portion thereof. FIG. 23 further shows that valve spool 196 includes one or more transfer ports 222, which extend from internal irrigation channel 213 to an outer surface 220 (best shown in FIGS. 24, 26). The transfer ports 222 are thus capable of allowing irrigation fluid (or therapeutic fluid) to flow into manifold 203, depending on the position of valve spool 196, as described in greater detail below. Valve surface 220 is configured in size and shape to mate with a corresponding inside surface 218 of manifold 203. When the distal end portion of valve spool 196 is fully introduced through bore 216 and into manifold 203, surface 220 fully engages and is seated against mating inside valve surface 218. This engagement seals the transfer ports 222, “closing” the valve, which blocks the flow of irrigation fluid. FIG. 24 is a cross-sectional view of catheter 10m and shows the valve spool 196 is the closed position.

However, when the valve spool 196 is moved in the proximal direction relative to electrode 192, the surface 220 disengages from mating surface 218, thereby unsealing transfer ports 222. Unsealing transfer ports 222 allows irrigation fluid to flow therethrough and into manifold 203. FIG. 26 is a cross-sectional view of catheter 10m, with the valve spool 196 in the open position.

Valve spool 196 includes various features that make use of the controlled movement provided by actuator coil 194 to facilitate selective opening and closing of the irrigation ports, as well to control movement to intermediate positions therebetween. In this regard, valve spool 196 includes a hub 224 having a distally-facing shoulder 226 and a proximally-facing shoulder 230. Likewise, ablation electrode 192 also includes a proximally-facing shoulder 228. Catheter 10m further includes a proximally-disposed buttress 232, which is shown in phantom-line format in FIG. 23.

With continued reference to FIG. 23, actuator coil 194 is disposed between shoulders 226, 228, while counter coil 198 is disposed between shoulder 230 and buttress 232. In a material phase change-based actuator embodiment, coil 194 has a first, non-activated state and a second, activated state, different from the first state in which the coil has a different mechanical characteristic. In FIGS. 23 and 24, coil 194 is in the first, non-activated state where the coil is in a compressed condition and has an axial length 234 (designated herein as L₂₃₄). The counter coil 198 urges valve spool 196 in the distal direction, using buttress 232 as a support. As shown, valve spool 196 is in its most forward position where all transfer ports 222 are sealed and thus the flow of irrigation fluid is blocked. This sealing is by virtue of valve surface 220 being engaged with and seated against inner manifold valve surface 218.

Referring now to FIGS. 25-26, the irrigation ports are in a fully opened condition. When delivery of irrigation fluid (or other fluid) is desired, controller 26 (not shown but described elsewhere) produces actuation input 24, which in this embodiment may be an activation signal, and to provide the activation signal to actuator coil 194. Once actuator coil 194 has been activated (e.g., thermally activated in one embodiment) through controller 26, the coil 194 transitions from a first state (compressed) to a second state (expanded), as described above. As shown in FIG. 25, actuator coil 194 now has an axial length 236 (designated L₂₃₆), which is greater than axial length 234 shown in FIG. 23. The expansion in the axial length of coil 194 urges valve spool 196 axially outwardly from electrode 192, wherein transfer ports 222 become unseated and are thus open to admit irrigation fluid into manifold 203. It should be understood that the force provided by the expansion of actuator coil 194 when activated should be selected so as to exceed that needed to overcome counter coil 198 (e.g., where the counter coil 198 is a conventional spring bearing against buttress 232). In other embodiments, where the counter coil 198 is also subject to activation (e.g., also a coil comprising SMA materials), controller 26 may be configured to sequence the respective deactivation and activation of actuator coil 194 and counter coil 198, respectively, so as to close the irrigation ports.

Finally, as best shown in FIG. 26, in a fully opened condition, both distal ports 204 and proximal ports 210 are open. However, variations in addition to fully opened and fully closed conditions are possible. For example, although not shown, actuator coil 194 may be activated so as to expand to a third state where the coil's axial length is in between axial length 234 and axial length 236, and sufficient to move the spool 196 to an intermediate position between fully open and fully closed (e.g., wherein only the distal irrigation ports 204 are open). In sum, the controlled movement (expansion and contraction) imparted by coil 194 to spool 196 is operative to open and close the irrigation ports disposed in and on electrode 192. It should be appreciated that the foregoing opening and closing of irrigation ports can occur without altering the mechanical characteristics of the main catheter body (shaft).

In further embodiments (not shown), an external feedback system may be used to control delivered flow rates, the location and direction of flow, all based on local conditions. For example, an irrigation fluid valve controlled or responsive to feedback may be based on conditions or parameters recorded during an ablation procedure or similar catheter-based therapeutic procedures. In a further embodiment, the irrigation ports need not be discrete, but rather may be perforated or formed using porous materials.

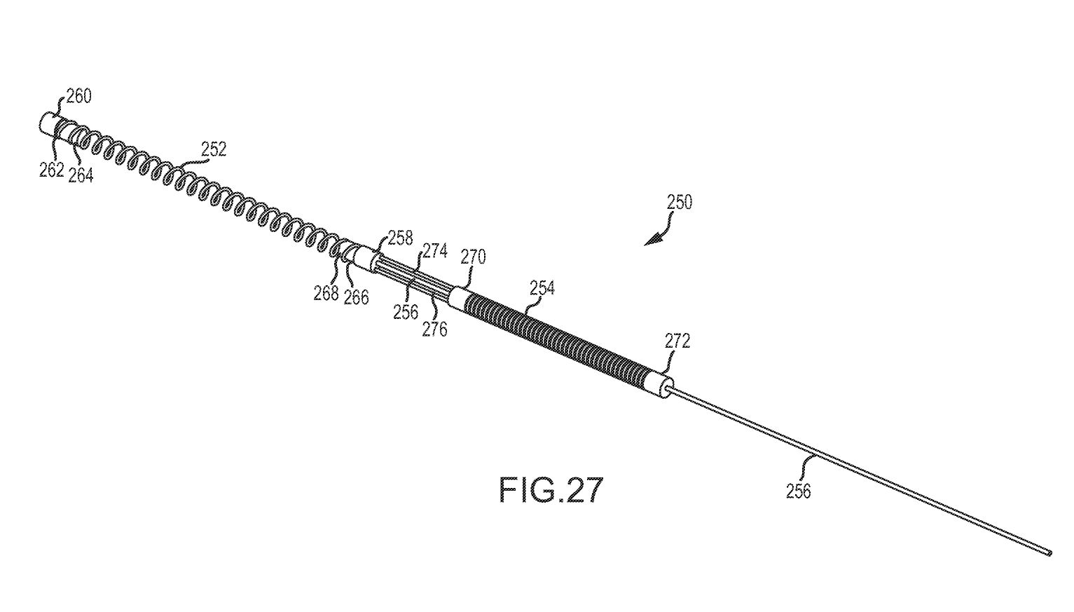

FIGS. 27-28, 29A-29C and 30-31 illustrate uses for further embodiments of remote actuation mechanism. As described above, NiTi coils may be heat treated to provide thermal recovery can provide force and displacement when subjected to temperatures above a transition temperature. In embodiments, multiple NiTi coils (or similar configurations) can provide localized force elements, deflection control and independent deployment capabilities. In addition, relatively high force to size ratios are possible.

FIG. 27 is an isometric view of a remote actuator 250 suitable for use in a catheter. The remote actuator 250 operates on the basis of a pair of counter-acting actuator coils wherein each one of the pair may operate based on being activated to change phase (e.g., NiTi coil or wire form). Remote actuator 250 may be configured in size so as to fit within an outer catheter shaft, for example, at a distal end portion of the catheter.

As shown, remote actuator 250 includes a load coil 252, a reset coil 254 and a pull wire 256. Load coil 252 is disposed between a guide 258 and a distal plug 260. Plug 260 includes a shoulder portion 262 and a reduced-diameter proximal portion 264 onto which load coil 252 is placed. A distal end of load coil 252 abuts shoulder 262 Likewise, guide 258 includes a shoulder 266 and a reduced diameter portion 268. The reduced diameter portion 268 is sized to accommodate an inner diameter of coil 252 wherein a proximal end of coil 252 engages shoulder 266. Reset coil 254 is disposed between guides 270 and 272. Guide 258 and guide 270 are coupled one with each other by a pair of radially spaced connecting rods 274, 276. For reference, the left-hand side of actuator 250 (as in FIG. 27) is the proximal end while the right-hand side of actuator is the distal end.

In the de-activated state, load coil 252, as shown, is in an distended configuration. Likewise, in the de-activated state, reset coil 254 is in a contracted configuration. Load coil 252 may be configured to provide a slightly greater retraction force (e.g., by using heavier wire and/or smaller diameter) than coil 254 to both deflect the catheter and extend coil 254 upon activation.

In the configuration shown, the outermost end of each NiTi coil 252, 254 are “grounded” mechanically speaking relative to the catheter shaft at respective ends 260, 272. Upon an input of energy to coil 252 (i.e., activation, for example, through the flow of electrical current through the coils or providing heat to the coils through a separate heater element), coil 252 will change phase and transition to a contracted state, thereby pulling coil 254 and pull wire 256 in a generally proximal direction. Although not shown, the distal end of pull wire 256 may be anchored distally to a pull ring or the like. The proximal end of pull wire 256 may be anchored in element 258 or, preferably, be free to move axially through element 258 until an end-of-travel limit is reached (e.g., a ball end or head on the pull wire engaging element 258). The tension developed when coil 252 contracts acts to deflect the catheter distal end portion (i.e., tip portion). Controller 26 may be configured, as described above, to modulate current flow through the load coil 252 (i.e., or control heater output) to provide varying degrees of coil activation and thus also varying degrees of catheter deflection. In an embodiment, both coils 252, 254 may be provided energy at varying levels to provide counter forces to help stabilize the catheter.

To return the catheter distal end portion (tip) to a non-deflected state, controller 26 activates reset coil 254 (i.e., deliver energy as described above). Coil 254, when activated through delivery of energy, changes phase and thus transitions from it current state (i.e., distended by virtue of the previous activation of load coil 252) to a contracted state, thereby pulling coil 252 into a contracted (closed) position through connectors 274, 276.

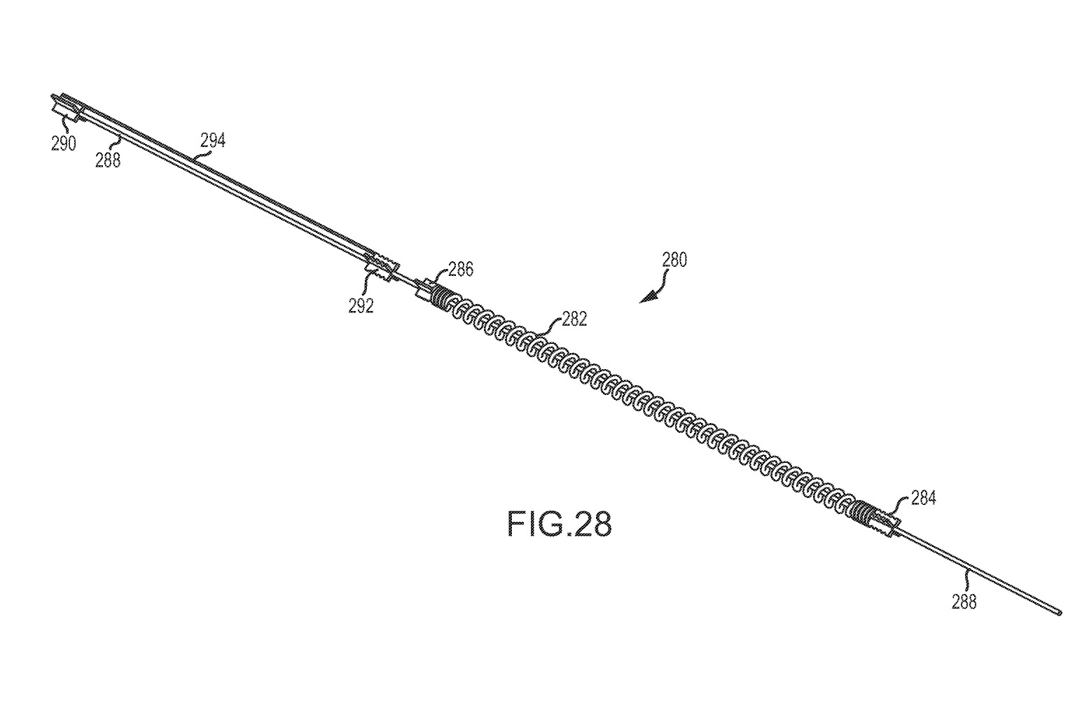

FIG. 28 is an isometric view of a remote actuator 280 suitable for use in a catheter. The remote actuator 280 operates on the basis of a single acting actuator coil that can be activated to change material phase, and thus also to change a mechanical characteristic of the coil such as its size (e.g., NiTi coil or wire form, as described above). Remote actuator 280 further includes a pull wire reset feature in lieu of a reset coil as was the case with remote actuator 250. Remote actuator 280 may be configured in size so as to fit within an outer catheter shaft, for example, at a distal end portion of the catheter.

Remote actuator 280 includes a load or actuator coil 282 disposed between buttresses 284 and 286. Actuator coil 282 is configured to be coupled to a controller, such as controller 26 described above, for selective activation. As noted, coil 282 has a first state (compressed) and a second, different state (extended) when activated. FIG. 28 shows coil 282 in the second (extended) state. The remote actuator 280 also includes a pull wire 288, which may be used to reset the actuator (i.e., return the remote actuator to an original state). Remote actuator 280 further includes first and second fixtures 290, 292 where first fixture 290 is located at an extreme distal end of the actuator 280 and second fixture 292 is located proximal of the first fixture 290. As shown, the pull wire 288 extends through coil 282, fixture 292 and is terminated to distal fixture 290. In addition, a beam 294 is disposed between fixtures 290, 292.

In FIG. 28, the left-hand side of actuator 280 is the distal end while the right-hand side if the proximal end. Features 284 and 292 are “grounded” to the catheter body (e.g., shaft). Wire 288 is fixed in elements 290 and 286 and is free to move axially in features 284, 290. Beam 294 provides a backbone for the remote actuator 280 that is deflected when a tensile force is supplied through or via wire 288 by extension of coil 282. Beam 294 may also provide a resetting force to help return the catheter tip to its un-deflected state. In an embodiment, additional resetting force may be provided through or by an operator input at a handle using wire 288. Extension of coil 282 to develop the above-described tensile force occurs through the phase change mechanism described herein. Specifically, controller 26 (not shown in FIG. 28) is configured to provide energy to the coil 282, which may comprise NiTi or similar SMA materials, for activation thereof. The controller may provide energy, for example, by providing an electrical current through the coil 282 or activating a separate heating element that is near coil 282. Controller 26 may be configured to manage the delivery of the energy to the coil, and may involve monitoring a coil temperature. The remote actuator 280 operates in tension as described above or via compression if a temperature induced phase change of the NiTi material causes the coil 282 to return to an extended state. In this latter case, the deflection would be in the opposite direction. Appropriate criteria to consider in selecting either the tension-based or the compression-based configuration may be the efficiency of the particular actuator configuration to deflect a catheter in a controlled manner.

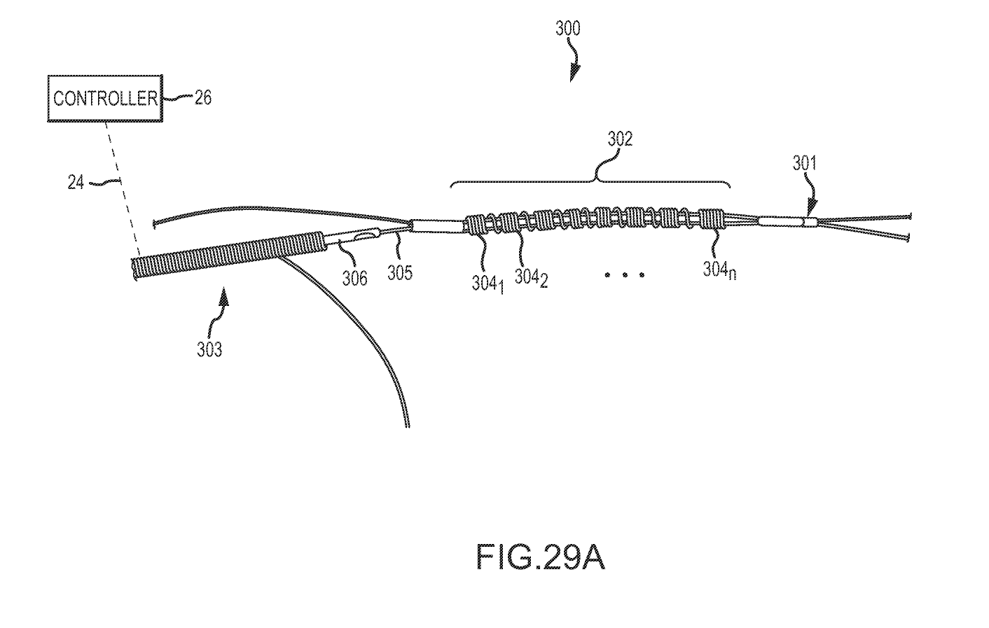

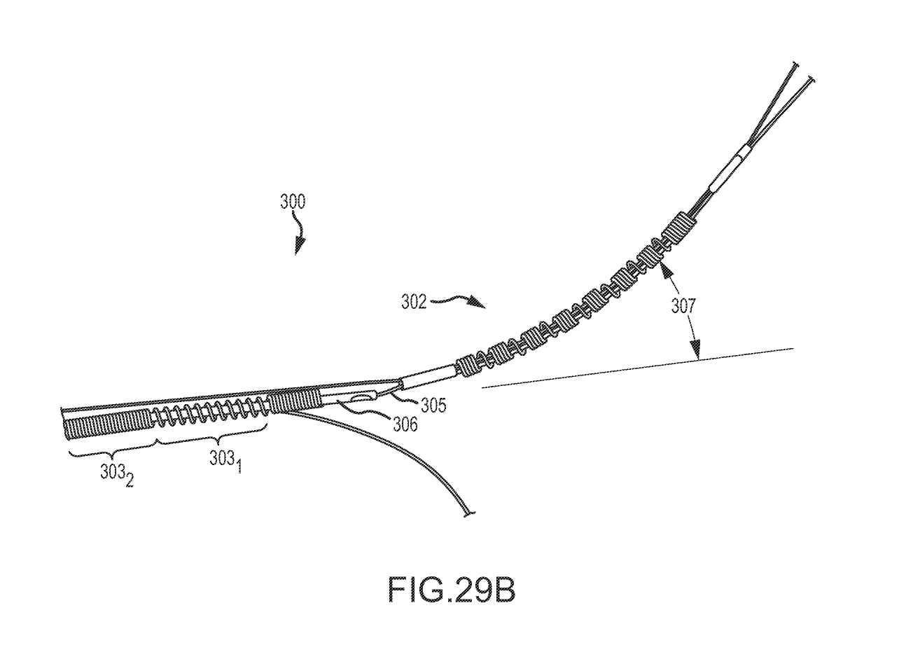

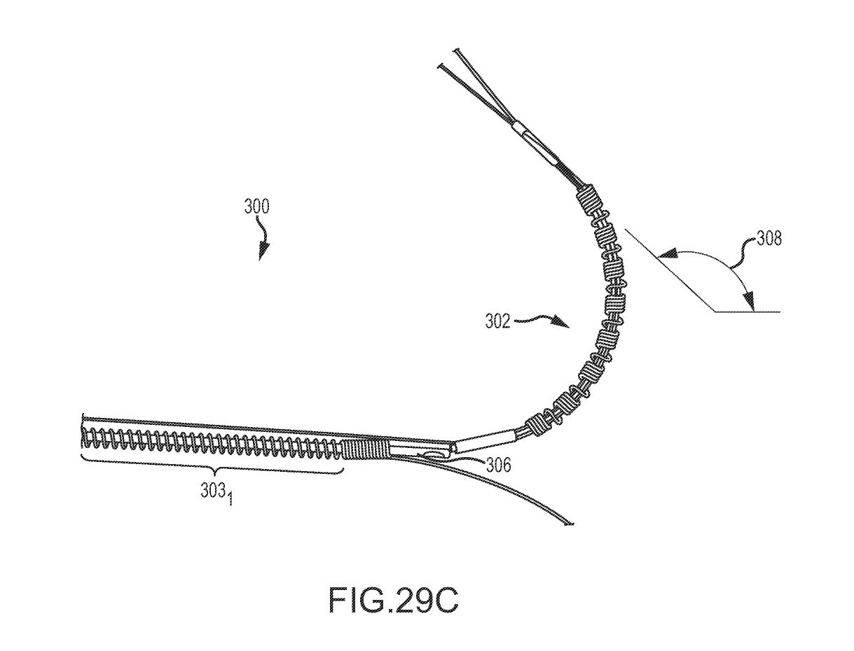

FIGS. 29A-29C are isometric, side views of a remote actuator 300 suitable for use in a catheter. Remote actuator 300 includes (i) a relatively flexible section 302 adapted for deflection including a plurality of coils 304₁, 304₂, . . . 304ₙ and (ii) a single-acting actuator coil 303, which may comprise a shape memory alloy (SMA) coil or wire. As described above, actuator coil 303 may comprise NITINOL material and phase change properties when thermally activated, as described above. Remote actuator 300 further includes a force transmitting member 305 fixed at a distal end 301 of remote actuator 300. Member 305 is further fixed at an extreme proximal end portion of coil 303. Coil 303 is disposed about a carrier 306. In effect, actuator coil 303 and carrier 306 operate, when activated, to “pull” on member 305 similar to applying a pull force to a pull wire to obtain remote deflection.

FIG. 29A further shows controller 26 coupled to actuator coil 303 and is configured to selectively assert an actuation input, which in this embodiment may be an activation signal (i.e., such as an electric signal so as to increase the temperature of coil 303, relying on thermal activation properties of the material used in coil 303). When actuator coil 303 is in a deactivated state, coil 303 is axially compressed, such that force transmitting member 305 is slack and portion 302 of remote actuator 300 assumes a relatively straight (un-deflected) configuration, as shown in FIG. 29A.

FIG. 29B shows a first stage of deflection. Controller 26 produces an actuation input, which for coil 303 is a thermal activation signal. The temperature of coil 303 rises, and once the temperature of the coil exceeds a transition temperature, coil 303 begins to transition from a first state to a second state wherein the second state exhibits a different physical or mechanical characteristic. For example, as shown in FIG. 29B, coil 303 begins to extend, as exemplified by coil portion 303₁, while the remainder portion 303₂ of coil 303 remains in a compressed state. The extension provided by coil portion 303₁ forces member 305 into tension, “pulling” on member 305 and thereby causing portion 302 to deflect, as shown. Since the wire that extends to the left from the tube section near feature 305 is attached to the opposite end of coil 303, when coil 303 extends during phase transformation a tensile force is applied to the element on the distal end causing deflection. The deflection wire runs under (and is constrained by) coil 302. As coil 303 extends the tensile force through the wire pulls the tip into a deflected arc. In an embodiment, within coil 302 is disposed a NiTi backbone that is intended to help form a smooth arc during deflection and to provide some return force. In FIG. 29B, the distal portion 302 of remote actuator 300 has deflected to about a first angle, designated angle 307.

FIG. 29C shows a still further stage where actuator coil 303 continues to expand wherein portion 303₁ thereof has increased significantly, thereby “pulling” member 305 to a greater extent, thereby resulting in an even greater level of deflection of portion 302. As shown, the deflection angle is represented by angle 308.

In a further embodiment, a pull wire reset feature is provided in actuator 300. In particular, such a pull wire may be integrated in a catheter wall and attached to the distal end 301 (FIG. 29A) or near distal end 301. The force provided by such a pull wire would supplement that return force already provided by the above-mentioned NiTi backbone.

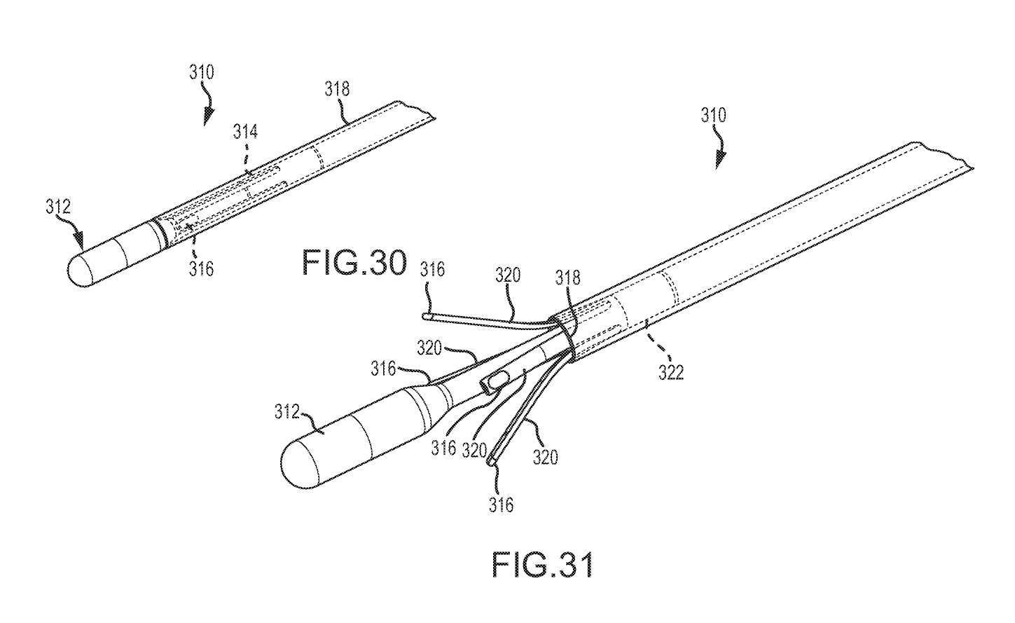

FIGS. 30-31 illustrate a catheter 310 with a remote actuation feature for remote deployment of a sensor array, shown in respective stowed and deployed positions. Catheter 310 includes a distal electrode 312, a three-dimensional electrode array 314 including a plurality of sensor electrodes 316, and an outer shaft 318.

The sensor electrodes 316 of array 314 are respectively disposed at the distal end portions of a plurality of arms 320 extending from a base collar 322. In the illustrated embodiment, four sensor electrodes 316 are respectively disposed on four arms 320.

Shaft 318 may be configured so as to be retractable (as shown in FIG. 31) or may be configured to retract automatically when array 314 is deployed (as described below). Array 314 may be deployed in three-dimensional space through the use of an actuation mechanism. In the illustrated embodiment, the actuation mechanism includes a configuration of the arms 320 to include shape memory alloy material (e.g., Nitinol material). Through application of an appropriate actuation input (e.g., an activation signal sufficient for thermal activation, in the case of a SMA-based actuation embodiment), the arms 320 will deflect away from a main axis of catheter 310, as shown in FIG. 31.

In view of the foregoing, it should be appreciated variations may be made to both remote actuation and low force pull wire embodiments described herein. For example, catheter embodiments described herein may be ablation catheters (i.e., either irrigated or non-irrigated), sensing catheters (i.e., either electrode or non-electrode based), catheters for an electro-anatomical mapping or other catheter types now known or hereafter developed. Although not shown, catheter embodiments, as known in the art, may be configured for use with external electronics to facilitate such functionality, and may comprise, in the case of a mapping catheter, visualization, mapping and navigation/localization components known in the art, including among others, for example, an EnSite Velocity™ system running a version of NavX™ software commercially available from St. Jude Medical, Inc., of St. Paul, Minn. and as also seen generally by reference to U.S. Pat. No. 7,263,397 entitled “METHOD AND APPARATUS FOR CATHETER NAVIGATION AND LOCATION AND MAPPING IN THE HEART” to Hauck et al., owned by the common assignee of the present invention, and hereby incorporated by reference in its entirety. Additionally, an electrophysiological (EP) monitor or display such as an electrogram signal display or other systems conventional in the art may also be coupled (directly or indirectly). Such an external localization system may comprise conventional apparatus known generally in the art, for example, an EnSite Velocity system described above or other known technologies for locating/navigating a catheter in space (and for visualization), including for example, the CARTO visualization and location system of Biosense Webster, Inc., (e.g., as exemplified by U.S. Pat. No. 6,690,963 entitled “System for Determining the Location and Orientation of an Invasive Medical Instrument” hereby incorporated by reference in its entirety), the AURORA® system of Northern Digital Inc., a magnetic field based localization system such as the gMPS system based on technology from MediGuide Ltd. of Haifa, Israel and now owned by St. Jude Medical, Inc. (e.g., as exemplified by U.S. Pat. Nos. 7,386,339, 7,197,354 and 6,233,476, all of which are hereby incorporated by reference in their entireties) or a hybrid magnetic field-impedance based system, such as the CARTO 3 visualization and location system of Biosense Webster, Inc. (e.g., as exemplified by U.S. Pat. No. 7,536,218, hereby incorporated by reference in its entirety). Some of the localization, navigation and/or visualization systems may involve providing a sensor for producing signals indicative of catheter location and/or orientation information, and may include, for example one or more electrodes in the case of an impedance-based localization system such as the EnSite™ Velocity system running NavX software, which electrodes may already exist in some instances, or alternatively, one or more coils (i.e., wire windings) configured to detect one or more characteristics of a low-strength magnetic field, for example, in the case of a magnetic-field based localization system such as the gMPS system using technology from MediGuide Ltd. described above.

Moreover, structures and arrangements for remote actuation and/or low force pull wire actuation, as described herein, may be readily incorporated with or integrated into catheter embodiments for performing ablative procedures. In this regard, it should be understood that such ablation catheter systems may, and typically will, include other structures and functions omitted herein for clarity, such as such as one or more body surface electrodes (skin patches) for application onto the body of a patient (e.g., an RF dispersive indifferent electrode/patch for RF ablation), and at least one irrigation fluid source (gravity feed or pump), an RF ablation generator (e.g., such as a commercially available unit sold under the model number IBI-1500T RF Cardiac Ablation Generator, available from St. Jude Medical, Inc.) and the like. Moreover, other types of energy sources (i.e., other than radio-frequency—RF energy) may also be used in connection with catheter 100, such as ultrasound (e.g. high-intensity focused ultrasound (HIFU)), laser, cryogenic, chemical, photo-chemical or other energy used (or combinations and/or hybrids thereof) for performing ablative procedures. Additional electrode tips may be used and configured, such as a closed loop cooled tip. Further configurations, such as balloon-based delivery configurations, may be incorporated into catheter embodiment consistent with the invention. Furthermore, various sensing structures may also be included in catheter 100, such as temperature sensor(s), force sensors, various localization sensors (see description above), imaging sensors and the like.

It should also be appreciated that while some catheter embodiments may be manually controlled, for example, through the use of a manually-actuated handle or the like, that robotically-actuated embodiments are also contemplated. For example, the controller 26 described above may be incorporated into a larger robotic catheter guidance and control system, for example, as seen by reference to U.S. application Ser. No. 12/751,843 filed March 31, 2010 entitled ROBOTIC CATHETER SYSTEM (Docket No.: 0G-043516US), owned by the common assignee of the present invention and hereby incorporated by reference in its entirety. A robotic catheter system may be configured to manipulate and maneuver catheter embodiments within a lumen or a cavity of a human body.

Briefly, such a robotic system may include a medical device, such as a catheter having the remotely-actuated functionality as described herein, a manipulator assembly, and a programmable electronic control unit (ECU). The medical device includes a shaft with a distal end portion and a remainder portion with the remainder portion including a proximal end portion. The device includes an actuator disposed at the distal end portion of the shaft. The actuator is configured to produce a controlled movement in response to an actuation input.

The manipulator assembly is configured to at least maneuver (e.g., advancing and withdrawing translation movement) the medical device, although in alternate embodiment the manipulator may also be configured to effectuate distal end (tip) deflection and/or rotation or virtual rotation. In further embodiments, the manipulator assembly may include actuation mechanisms (e.g., electric motor and lead screw combination) for linearly actuating one or more control members associated with the medical device for achieving the above-described translation, deflection and/or rotation (or virtual rotation). It should be understood, however, that the spirit and scope of the inventions contemplated herein is not so limited and extends to and covers, for example only, a manipulator assembly configured to employ rotary actuation of the control members.

The electronic control unit (ECU) is coupled to the manipulator assembly for control thereof in response to operator inputs and in accordance with a predetermined operating strategy. The ECU is configured (e.g., through software stored in a memory accessible by the ECU) to either generate the actuation input destined for the remote actuator (e.g., where the actuation input comprises an electric signal) or to cause the actuation input to be generated (e.g., where the actuation input is a hydraulic pressure signal). The ECU is disposed in a location remote from the distal end portion and wherein the actuation input is communicated to the distal remote actuator without altering the mechanical characteristics of the remainder portion of the shaft. Through the foregoing, one or more of the disadvantages of the conventional art may be overcome.

In further embodiments, the ECU is configured to cause the manipulator assembly to either linearly actuate and rotary actuate one or more control members associated with the medical device for at least one of translation and deflection movement.

The embodiments disclosed and depicted herein may be fabricated using known approaches and conventional materials, in accordance with the description contained herein. In further embodiments that incorporate a material phase change based actuator (e.g., as described above), a method of fabricating a medical device having remote-actuated distal end functionality includes a number of steps.

The first step involves configuring a structure (e.g., a coil or wire form) comprising the shape memory alloy (SMA) material into a predetermined shape. In the method, the predetermined shape may be the desired activated shape of the structure, as that term is used herein. The next step involves heating the structure (in the desired, predetermined shape) above a transition temperature associated with the SMA material being used so as to heat set the structure. In one embodiment, the heat set structure is then allowed to cool before further fabrication steps are performed. In another embodiment, however, the step of heat setting the SMA structure may be performed while the SMA structure is already incorporated into an actuator design. For example, in the embodiment illustrated in FIGS. 23-26, the SMA-based actuator (i.e., actuator coil 194 and optionally counter coil 198) may be assembled onto electrode 192 and valve spool 196 and then configured into the desired, activated shape (e.g., for actuator coil 194, the activated shape is an extended coil) and then heat set. In the former method of fabrication embodiments, the method then further involves the step of incorporating the heat set structure into an actuator and disposing the actuator in a distal end portion of the medical device.

The method may also involve providing a means for communicating an actuation input from a proximal end of the medical device to the actuator disposed at the distal end. For example, the actuation input may be an electrical activation signal that is directed through the SMA structure or to an adjacent heater to provide heat to the SMA structure. The method may also involve encapsulating the SMA actuator and the means for communicating the actuation input, so as to isolate and protect the actuator and communication means for environmental factors typically encountered by a medical device. In this regard, the encapsulating step may include the sub-step of subjecting the medical device to a reflow lamination process.

Depending on the type of medical device being fabricated, a cylindrical or specially-shaped mandrel of a desired length may be used (e.g., over which liner materials, reaction coils, structural tubes, hollow core materials, steering wires or other materials and structures described herein or as known in the art may be placed). For example, an outer layer of relatively flexible polymer material (e.g., PEBAX material) may be placed over a sub-assembly which itself is disposed over a mandrel. Such an outer layer may comprise either a single section or alternatively multiple sections of tubing that are either butted together or overlapped with each other. The multiple segments, or layers, of sheath material may be any length and/or hardness (durometer) allowing for flexibility of design, as known in the art. Such an assembly thus formed is then subjected to a reflow lamination process, which involves heating the assembly until the outer layer material flows and redistributes around the circumference. The device is then cooled. The distal and proximal end portions of such a device may then be finished in a desired fashion.

Generally, except as described above with respect to the various embodiments, the materials and construction methods for manufacture of a medical device may comprise corresponding conventional materials and construction methods, for example only, as seen by reference to U.S. patent application Ser. No. 11/779,488 filed Jul. 18, 2007 entitled CATHETER AND INTRODUCER CATHETER HAVING TORQUE TRANSFER LAYER AND METHOD OF MANUFACTURE, owned by the common assignee of the present invention and hereby incorporated by reference in its entirety. It should be understood that the foregoing are exemplary only, and not limiting in nature, inasmuch as other known approaches for fabrication may be used, other materials may be used and component dimensions may be realized.

It should be understood that an electronic controller or ECU as described above for certain embodiments may include conventional processing apparatus known in the art, capable of executing pre-programmed instructions stored in an associated memory, all performing in accordance with the functionality described herein. To the extent that the methods described herein are embodied in software, the resulting software may be stored in an associated memory and where so described, may also constitute the means for performing such methods. Implementation of certain embodiments of the invention, where done so in software, would require no more than routine application of programming skills by one of ordinary skill in the art, in view of the foregoing enabling description. Such an electronic controller or ECU may further be of the type having both ROM, RAM, a combination of non-volatile and volatile (modifiable) memory so that the software can be stored and yet allow storage and processing of dynamically produced data and/or signals.

In a further embodiment, a medical device such as an RF ablation catheter includes an irrigation fluid valve that is actuated automatically by a heat activated SMA-based actuator (e.g., based on a rise in temperature of the actuator due to heat dissipated during RF ablation). The actuation of such a valve may operate to alter the path of irrigation fluid within the catheter, which may increase or decrease the volume of fluid delivered, change the pattern of irrigation, for example, from one port to another port, from one side of the ablation electrode (e.g., the non-contact side when starting an ablation procedure) to the other side (e.g., the contact side of the electrode when the local temperature has increased above a transition threshold), from distal tip irrigation ports to proximal irrigation portion, from one non-contact side of the electrode (e.g., the left side) to the other non-contact side of the electrode (e.g., the right side), or other irrigation pattern. The actuator in this embodiment may comprise an SMA-based structure configured to impart a controlled movement to actuate the fluid valve when the SMA-based structure reaches its designed transition temperature, as described more generally above. The SMA-based structure in this embodiment may be activated using local tissue and/or ablation electrode temperature increases arising from dissipated heat attendant the RF ablation procedure per se, as opposed to a dedicated circuit configured to deliver an actuation input, such as an electrical signal, directly to the SMA-based structure or to an adjacent heater. This embodiment has the benefit of not requiring additional, dedicated control or activation wires (e.g., typically 1 or 2 wires) or circuitry for activation of the actuator (SMA-based structure). Rather, the existing wiring for RF energy delivery is used, in effect, to cause (indirectly via heat activation) the actuation of the fluid valve to occur. In an embodiment, an optional, resistive element may be added to the catheter near the distal end, in the RF power delivery circuit (e.g., series, parallel), in order to increase, by a predetermined measure, the amount of heat dissipated during an RF ablation procedure. This optional feature may be used, for example, to hasten the timing of the activation of the SMA-based actuator, as compared to the timing without the resistive element. This feature may be used to actuate the irrigation fluid valve and thus alter the pattern of irrigation at an earlier time compared to the same SMA-based actuator/valve combination without the resistive element installed. Such an element may be a serpentine heating element, a variable resistor, a surface mount technology (SMT) resistor, or other technology for resistive heating now know or hereafter developed. Conversely, the timing of the activation of the SMA-based actuator, in this embodiment, may be delayed by including a cooling mechanism, such as a Peltier cooler (thermo-electric element) for thermo-electric cooling. In this alternative, the thermo-electric element is disposed in or near the distal end portion of the ablation catheter and is configured, relative to the RF power delivery circuit (e.g., series, parallel), for endothermic operation so as to draw heat away (i.e., thermal sink) for the site and/or device and thus delay the increase in the temperature of the SMA-based actuator to its transition temperature.

In a further embodiment, a medical device, such as a catheter, includes a heat-activated SMA-based actuator configured to close or open an electrical switch, for example, for redistributing electrical RF energy among and/or between multiple electrodes in a multi-electrode ablation catheter. This embodiment also has the benefit of not requiring additional, dedicated control or activation wires (e.g., typically 1 or 2 wires) or circuitry for activation of the actuator (SMA-based structure). Rather, the existing wiring for RF energy delivery is used, in effect, to indirectly cause (i.e., through heat activation) the electrical switch to be opened or closed.

In a further embodiment, in any of the heat-activated embodiments, an external electronics module is configured with a notification means (e.g., circuitry, software) to provide clinician notification (e.g., audio, visual, such as yellow/slow down and red/stop, or haptic feedback) when the SMA-based structure is nearing or has reached its transition temperature (and thus when the designed actuation is about to occur, e.g., irrigation pattern adjustment and/or RF energy redistribution).

In a further embodiment, a medical device may be configured with an SMA-based actuator having a plurality of transition temperatures. For example, an irrigation fluid valve arrangement may be configured so that at normal body temperature (37 degrees C.), the fluid valve is “closed” or otherwise substantially divert irrigation fluid away from the ablation site. When the ablation site reaches a first transition temperature (e.g., 45 degrees C.), the fluid valve is actuated by the SMA-based structure by a first amount or to a first position so as to allow “half” rated irrigation fluid flow near the ablation electrode/ablation site. When the ablation electrode/ablation site reaches a second transition temperature (e.g., 60 degrees C.), the fluid valve is actuated by the SMA-based structure by a second amount or to a second position so as to allow “full” rated irrigation fluid flow. In a still further embodiment, the above-described cooling mechanism (i.e., thermo-electric element) is used so as to decrease the body temperature by a predetermined amount (e.g., to 35 degrees C.) before the RF ablation procedure is begun.

Although a number of embodiments of this invention have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A catheter comprising:
a shaft having a distal end portion and a remainder portion including a proximal end portion;
an actuator disposed at said distal end portion of said shaft, said actuator being configured to produce a controlled movement responsive to an actuation input;
a controller configured to produce said actuation input, said controller being disposed in a location remote from said distal end portion and wherein said actuation input is communicated to said distal remote actuator without altering the mechanical characteristics of said remainder portion of said shaft.

2. The catheter of claim 1 wherein said mechanical characteristics include a deflection characteristic.

3. The catheter of claim 1 wherein said actuator is one of a magnetic actuator, material phase-change actuator, a hydraulic actuator, a piezo-electric actuator, electromagnet actuator, permanent magnet actuator, a solenoid, and an electric actuator.

4. The catheter of claim 3 wherein said actuator comprises said material phase-change actuator, including at least one NiTi coil configured to transition, responsive to said input signal, from a first state to a second, different state having a different physical configuration.

5. The catheter of claim 1 wherein said controlled movement is configured for one of deflection of said distal end portion of said shaft, sensor deployment, control of fluid delivery rate, control of fluid delivery direction, control of fluid delivery location, deployment of catheter retention mechanism.

6. The catheter of claim 5 wherein said remote actuator is configured to control a fluid valve, said valve being configured to control one of said fluid delivery rate and said fluid delivery direction.

7. The catheter of claim 1 wherein said controller is configured to vary said actuator input signal in accordance with changes in one or more of a condition or a parameter, said remote actuator being responsive to said varying input signal to thereby vary said controlled movement.

8. The catheter of claim 1 wherein said actuator is coupled to a deflection mechanism disposed at said distal end.

9. The catheter of claim 8 wherein said shaft includes an axis and wherein said deflection mechanism includes a spring assembly comprising a plurality of springs extending along said axis, each spring having a respective open end thereof, said mechanism further including an elongate member having a first end fixed at a first axial end of said spring assembly, said elongate member passing through said springs proximate said open ends and extending out of a second axial end of said spring assembly, a second end of said elongate member coupled to said actuator, said actuator being configured to impart said controlled movement to said elongate member, thereby causing said spring assembly to deflect.

10. The catheter of claim 9 wherein said springs are one of U-shaped and V-shaped.

11. The catheter of claim 9 wherein said springs of said spring assembly comprise a resilient material disposed in respective recesses thereof, said resilient material being compressed when said actuator causes said spring assembly to deflect, said compressed resilient material being configured to return said spring assembly to an original, non-deflected state when said actuator discontinues a tensile force on said elongate member.

12. The catheter of claim 9 wherein each spring of said spring assembly has a respective width which is compressed during deflection, said respective widths being one of substantially the same with respect to said plurality of springs and variable with respect to said plurality of springs.

13. The catheter of claim 9 wherein said deflection mechanism is a first deflection mechanism, said catheter including a plurality of deflection mechanisms.

14. The catheter of claim 9 further including a mechanism for adjusting an axial location of said deflection mechanism within said distal end portion of said shaft to thereby adjust the axial location where said shaft is deflected.

15. The catheter of claim 8 wherein said shaft includes an axis and wherein said deflection mechanism includes a bellows assembly comprising a plurality of chambers extending along said axis, each chamber being in fluid communication with an adjacent one of said chambers, said mechanism further including an elongate anchor member having a first end fixed at a first axial end of said bellows, said elongate anchor member passing through said chambers at a transverse side of said chambers, said elongate anchor member being fixed at a second axial end of said bellows assembly, said actuator being configured to deliver fluid to said bellows assembly at said first axial end to thereby causing said bellows assembly to deflect.

16. The catheter of claim 8 further comprising an elongate member extending within said shaft and having a proximal end at said shaft proximal end portion and a distal end coupled to said actuator, said elongate member being substantially coaxial with said shaft.

17. The catheter of claim 16 wherein said actuator is configured to translate a first tensile force that is transmitted via said elongate member along a first axis to a second tensile force that is transmitted via a force transmitting member along a second axis offset from said first axis.

18. The catheter of claim 17 wherein said actuator comprises a guide configured to maintain said elongate member substantially coaxial, wherein said distal end of said elongate member is affixed in said catheter at a point that is transversely offset from said first axis.

19. The catheter of claim 18 further including a resilient spar axially extending between said guide and a ring wherein said ring is disposed axially distal of said guide, said spar is configured to deflect from a first state to a second state when said first force is applied to said elongate member, said spur being configured to provide a restorative force when said first force is discontinued.

20. The catheter of claim 19 wherein said spur, when taken in axial cross-section, is arranged in one of a diagonal orientation and a horizontal orientation offset from said first, shaft axis.

21. The catheter of claim 19 further including a coil spring between said guide and said ring.

22. The catheter of claim 20 wherein said spring is tapered.

23. The catheter of claim 18 further comprising a second elongate member.

24. The catheter of claim 8 wherein said deflection mechanism comprises a guide, a spar extending distally from said guide, and a ring coupled to a distal end of said spar, said mechanism further including a force transmitting member having one end coupled to said actuator wherein the other end of said force transmitting member is coupled to said ring at a point that is transversely offset from a first axis of said shaft.

25. The catheter of claim 6 wherein said actuator comprises a structure that is configured to transition, responsive to said input signal, from a first state to a second, different state having a different physical configuration.

26. The catheter of claim 25 wherein said structure includes a coil arrangement comprising material whose phase is changed between said first and second states.

27. The catheter of claim 26 wherein said material comprises NiTi.

28. The catheter of claim 6 further including an electrode assembly including an ablation electrode at said distal end of said shaft, said electrode including at least one irrigation passageway extending between a manifold and an irrigation port on a surface of said electrode, said valve comprising a valve spool in communication with a fluid tube for delivery of irrigation fluid, said spool being coupled to said actuator for controlled movement between a closed position and an open position in which irrigation fluid is permitted to flow from said tube into said manifold.

29. The catheter of claim 28 wherein said valve spool includes an internal chamber in communication with said tube, said valve spool including a distal head configured to engage a complementary inner surface of said manifold, said distal head includes at least one transfer port extending between said chamber and an outer surface of said distal head, wherein when said valve spool is in said closed position, said transfer port abut said complementary inner surface of said manifold to thereby prevent fluid flow through the transfer port into said manifold and wherein when said valve spool is in said open position, said distal head is moved away from engagement with said complementary inner surface to thereby allow fluid to flow out of said transfer port into said manifold.

30. The catheter of claim 29 wherein said valve spool includes a first shoulder and wherein said electrode assembly includes a body portion having a second shoulder, said actuator including a coil arrangement disposed between said first and second shoulders, said coil arrangement comprising material configured to transition in material phase, responsive to said input signal, from a first state to a second, different state having a different physical configuration.

31. The catheter of claim 30 wherein said actuator further includes a counter member configured to return said valve spool to said closed position.

32. The catheter of claim 8 wherein said deflection mechanism comprises:
a first fixture;
a second fixture coupled to said first fixture by a backbone and wherein said second fixture is fixed relative to said shaft; and
a tensile member fixed to said first fixture and slidably coupled to said second fixture;
wherein said controlled movement of said actuator is configured to axially move said tensile member to thereby cause said deflection mechanism to deflect said catheter distal end portion.

33. The catheter of claim 32 wherein said remote actuator comprises:
a first buttress;
a second buttress fixed relative to said shaft;
a material phase change coil between said first and second buttresses, said tensile member is coupled to said first buttress;
said material phase change coil having expanded and extended states so as to axially move said tensile member.

34. The catheter of claim 1 wherein said actuator:
a plug fixed relative to said shaft;
a first guide;
a load element comprising a first material phase change coil between said plug and first guide;
a second guide coupled to said first guide by a connecting member;
a third guide fixed relative to said shaft;
a reset element comprising a second material phase change coil between said second guide and said third guide;
a tensile member having a proximal end fixed to said first guide, said member extending axially in a distal direction through said second and third guides, a distal end of said tensile member being configured for coupling to a deflection mechanism;
said load element having a first, de-activated state where the load element is axially extended, said load element having a second, activated state where the load element is axially contracted relative to said first state;
said reset element having a first, deactivated state where the reset element is axially contracted, said reset element having a second, activated state where the reset element is axially extended relative to said first state;
wherein when said load element is activated and said reset element is de-activated, said distal end of said tensile member moves in a proximal direction; and
wherein when said load element is de-activated and said reset element is activated, said load element is extended into said first state, thereby releasing said tensile member.

35. A robotic system for maneuvering a medical device, comprising:
a medical device including a shaft with a distal end portion and a remainder portion, said remainder portion including a proximal end portion, said medical device including an actuator disposed at said distal end portion of said shaft, said actuator being configured to produce a controlled movement responsive to an actuation input;
a manipulator assembly configured to at least maneuver said medical device;
an electronic control unit (ECU) coupled to said manipulator assembly for control thereof, said ECU being configured for one of generating said actuation input and causing said actuation input to be generated, said ECU being disposed in a location remote from said distal end portion and wherein said actuation input is communicated to said distal remote actuator without altering the mechanical characteristics of said remainder portion of said shaft.

36. The system of claim 35 wherein said controller is configured to cause said manipulator assembly to actuate one or more control members associated with said medical device in one of a linear fashion and a rotary fashion for effecting at least one of translation and deflection of said medical device.

37. A method of fabricating a medical device having distal end functionality, comprising the steps of:
configuring a structure comprising shape memory alloy (SMA) material into a predetermined shape;
heating the structure as configured in the predetermined shape above a transition temperature associated with the SMA material so as to heat set the structure;
incorporating the heat set structure into an actuator disposed in a distal end portion of the medical device; and
providing means for communicating an actuation input from a proximal end of the medical device to the actuator.

38. The method of claim 37 further comprising the steps of:
encapsulating the actuator and the means for communicating the actuation input to the actuator.

39. The method of claim 38 wherein said encapsulating step includes the sub-step of subjecting the medical device to a reflow lamination process.

40. A catheter comprising:
a shaft having a proximal end portion and a distal end portion;
a heat-activated actuator disposed at said distal end portion of said shaft, said heat-activated actuator being configured to produce a controlled movement when said actuator reaches a transition temperature, wherein said actuator is configured to actuate one of an electrical switch and a fluid valve.

41. The catheter of claim 40 wherein said catheter is a radio-frequency (RF) ablation catheter having a plurality of ablation electrodes, said switch having an open position where RF energy is delivered to a first pattern of said plurality of ablation electrodes, said switch having a closed position where said RF energy is delivered to a second pattern of said plurality of electrodes different from said first pattern, said heat-activated actuator being arranged in relation to said switch to change said switch between said open and closed positions based on whether said actuator temperature has reached said transition temperature.

42. The catheter of claim 40 wherein said catheter is a radio-frequency (RF) ablation catheter having a plurality of irrigation fluid patterns, said fluid valve having a first position configured to deliver irrigation fluid according to a first one of said plurality of irrigation patterns, said valve having a second position different from said first position where said valve is configured to deliver irrigation fluid according to a second one of said plurality of irrigation patterns, said heat-activated actuator being arranged in relation to said fluid valve to change said valve between said first and second positions based on whether said actuator temperature has reached said transition temperature.

43. The catheter of claim 40 wherein said actuator comprises a shape memory alloy (SMA) structure.
